(19) United States
(12) Patent Application Publication
(10) Pub. No.: US 2025/0279201 A1
(43) Pub. Date: Sep. 4, 2025

(54) CELL-FREE DNA-BASED METHODS

(71) Applicant: University of Essex Enterprises Limited, Colchester, Essex (GB)

(72) Inventor: Vladimir TEIF, Colchester, Essex (GB)

(21) Appl. No.: 18/858,173

(22) PCT Filed: Apr. 18, 2023
(86) PCT No.: PCT/GB2023/051019

§ 371 Date: Oct. 18, 2024

(30) Foreign Application Priority Data
Apr. 19, 2022 (GB) 2205710.3

(51) Int. Cl. G16H 50/20; G16B 5/00; G16B 20/20; G16B 30/10; G16B 40/10; G16B 40/20; G16H 10/60; G16H 50/70

(52) U.S. Cl. CPC G16H 50/20; G16B 5/00; G16B 20/20; G16B 30/10; G16B 40/10; G16B 40/20; G16H 10/60; G16H 50/70

(57) ABSTRACT

Aspects of the present invention relate at least in part to methods and systems for determining distribution of genomic distances between fragments of cell-free nucleic acids, which reflect the distribution of biomolecular complexes such as nucleosomes that protect genomic DNA from nuclease digestion, as well as different fractions of DNA fragments mapped to genomic DNA sequence repeats. Particularly, although not exclusively, embodiments of the present invention relate to a method for determining the distribution of distances between neighbouring nucleosomes, wherein said distribution of distances vary between diseased and healthy states. Aspects of the present invention comprise diagnostics, stratification and monitoring of subjects suffering from a disease or identification of different characteristics such as the subject's age.

### FIELD OF THE INVENTION

Aspects of the present invention relate at least in part to methods and systems for determining distribution of genomic distances between fragments of cell-free nucleic acids, which reflect the distribution of biomolecular complexes such as nucleosomes that protect genomic DNA from nuclease digestion, as well as different fractions of DNA fragments mapped to genomic DNA sequence repeats. The distribution of said distances may be genome-wide or may be within regions of interest in a portion of the genome on one or more chromosomes. In certain aspects, the invention provides methods and systems for determining nucleosome positioning based on cell-free nucleic acids in liquid biopsies. Particularly, although not exclusively, embodiments of the present invention relate to a method for determining the distribution of distances between neighbouring nucleosomes, wherein said distribution of distances vary between diseased and healthy states, as well as between different time-points for the same patient. In certain aspects, the invention provides a method for diagnostics based on the relative numbers of DNA fragments mapped to different types of genomic DNA sequence repeats, wherein said numbers vary between diseased and healthy states, as well as between different time-points for the same patient. In certain embodiments, the method determines whether a subject has a disease. Aspects of the present invention comprise diagnostics, stratification and monitoring of subjects suffering from a disease or identification of different characteristics such as the subject's age.

### BACKGROUND TO THE INVENTION

The term “liquid biopsy” encompasses the analysis of disease-associated biomarkers in the blood plasma, urine or other body fluids. For example, circulating cell-free DNA (cfDNA) is present in liquid biopsies and consequently the experimental procedure of cfDNA extraction is relatively simple, especially compared to the procedures of more traditional biopsies of tissues. Thus liquid biopsies can be used in methods of patient diagnostics, monitoring and stratification.

Current cfDNA analysis assays—based, for example, on gene mutations or DNA methylation analysis of a limited number of genes—have limitations in detecting disease-specific changes beyond a selected gene panel. The cost of whole-genome sequencing is currently limiting the mass-use of liquid biopsy assays based on deep-sequencing of total cfDNA. On the other hand, shallow whole-genome sequencing provides small sequencing coverage which makes it difficult to robustly detect mutations or DNA methylation changes. This is especially challenging in early disease stages, where the amount of disease-specific cfDNA is relatively low. The sensitivity of such methods depends on the sequencing depth as well as on the abundance of cfDNA derived from tumour cells (ctDNA), which usually correlates with the severity/stage of disease—and so may not be prevalent at the onset of disease. Unfortunately, not only does the requirement of at least moderate sequencing depth drive up the cost of such assays, but the timescale associated with accumulation of mutations or chemical modification can preclude the assay's efficacy in early-stage diagnosis. On the other hand, the turnover of cfDNA in blood is at the timescale of minutes, and cfDNA extracted from a patient's blood plasma at any given time point provides a recent representation of nucleosome positioning in the cells of origin. Therefore, the analysis of cfDNA fragments may not only be useful for diagnostics, but also in monitoring disease progression and a patient's response to therapy.

cfDNA is formed by pieces of DNA from many different cells. In each of these cells, the nucleases, which shred chromatin, can only cut genomic DNA between regions protected by nucleoprotein complexes such as nucleosomes. In the case of nucleosome-dependent digestion, this means that while linker DNA is digested out, the nucleosomal DNA is released to bodily fluids in the form of cfDNA fragments. Consequently, cfDNA fragments reflect areas of the genome protected by nucleosomes in the living cells, from which the cfDNA fragments originated. It therefore follows that analysis of the genomic maps of cfDNA fragments provides information on the nucleosome positioning landscapes in the cells of origin. It is well established that genomic nucleosome positions are associated with the activity of many biological processes, particularly biological processes that require access of regulatory molecules to the DNA.

Genomic maps of cfDNA fragments may also reflect the distribution of cfDNA fragments mapped to different genomic DNA sequence repeats, and the number of fragments mapped to such repeats may change in a disease. However, the straightforward comparison of genome-wide cfDNA maps is not very effective for diagnostics because of large degree of noise and stochasticity, as well as the dependence of such maps on the specific protocol of cfDNA extraction and sequencing coverage.

There are a number of computational methods for cfDNA analysis that focus on the analysis of cfDNA fragments per se (e.g., their properties and genomic locations of origin). These include e.g. the distribution of cfDNA fragment sizes or their nucleotide patterns, the density of cfDNA fragments in certain genomic regions, or the correlation of cfDNA patterns with the corresponding gene expression in previously acquired datasets (in different cell types).

For example, WO2021/130356A1 relates to methods of cell-free DNA (cfDNA) analysis based on cfDNA methylation, cfDNA copy number alteration and cfDNA nucleosome footprinting.

US2019/352695A1 and WO2022/040163A1 relate to methods for fragmentome profiling of cfDNA. In particular the methods disclosed in US2019/352695A1 and WO2022/040163A1 are based on analysis of cfDNA fragment sizes.

US2019/341127A1 relates to methods of analysing cfDNA using size-tagged preferred ends and orientation-aware analysis. In particular the methods disclosed in US2019/341127A1 are based on the fragmentation patterns of cfDNA (e.g., sizes of fragments).

WO2016/015058A2 and Synder et al (2016, Cell 164, 57-68) relate to methods of determining tissues and/or cell types giving rise to cell free DNA (cfDNA) in a subject. The methods disclosed in WO2016/015058A2 and Synder et al are based on a correlation between cfDNA maps and gene expression. In particular the disclosed methods involve calculating inter-nucleosome distances in regulatory regions associated with genes to infer cell types contributing to cfDNA in pathological states (such as cancer).

Markus et al (2021, Sci. Transl. Med. 13, eaaz3088) relates to methods of analysing recurrently protected genomic regions (RPR) in cfDNA. In particular the methods disclosed in Markus et al involve characterising cfDNA fragments based on the distance of fragments' start and end sites relative to their nearest RPR.

Shtumpf et al (2022, Chromosoma 131:19-28) discusses the NucPosDB database. In particular Shtumpf et al discloses an association between cfDNA CG content profile (as a function of the distance from the end of the cfDNA fragment) and medical conditions and association between profile of distribution of lengths of cfDNA fragments and medical conditions.

However, the challenge to provide good sensitivity and specificity required for widespread clinical use remains open. One possible reason for this is that parameters such as distributions of cfDNA sizes, cfDNA fragments' nucleotide patterns and gene expression are heterogeneous within a cohort of people with the same condition, as well as between cohorts assessed in different laboratories with different cfDNA extraction protocols. Another reason is that the analysis aiming to determine the cells of origin of cfDNA in body fluids may be problematic because cfDNA originates from many different cell types. At early disease stages, cfDNA originating from the cells representing a disease (e.g. tumour) can be a minority in the total cfDNA. Therefore, new methods are needed which do not rely on cfDNA fragment sizes and do not require explicitly determining the cells of origin of cfDNA.

It is an aim of certain embodiments of the present invention to at least partially mitigate the problems associated with the prior art.

It is an aim of certain embodiments of the present invention to provide a method to determine a subject's characteristics of the distribution of distances between cfDNA genomic locations that allow robust subject classification. Aptly such characteristics of the distribution of distances between cfDNA genomic locations may refer to the distribution of distances between genomic DNA regions protected from nuclease digestion. Aptly, the distribution of distances between genomic DNA regions protected from nuclease digestion may refer to a limited set of genomic regions (e.g., a chromosome or a region of interest or a set of regions) or across the genome as a whole.

It is an aim of certain embodiments of the present invention to provide a method to identify disease-specific changes in the distribution of distances between genomic DNA regions protected from nuclease digestion. Aptly disease-specific changes may refer to changes within a localised region or across the genome as a whole.

It is an aim of certain embodiments of the present invention to provide a method to diagnose disease based on a subject's distribution of distances between genomic DNA regions protected from nuclease digestion.

It is an aim of certain embodiments of the present invention to provide a method to diagnose, stratify and monitor patients based on a subject's distribution of distances between genomic DNA regions protected from nuclease digestion.

It is an aim of certain embodiments of the present invention to provide a method to diagnose disease based on a subject's distribution of relative numbers of cell-free DNA fragments mapped to different types of genomic sequence repeats.

It is an aim of certain embodiments of the present invention to provide a method to diagnose, stratify and monitor patients based on a subject's distribution of relative numbers of cell-free DNA fragments mapped to different types of genomic sequence repeats.

### SUMMARY OF CERTAIN EMBODIMENTS OF THE INVENTION

There remains a clear need to develop a method to determine the mathematical characteristics of cfDNA which are robust with respect to different cfDNA extraction protocols and allow effective subject classification as well as comparison of cfDNA samples taken from the same subject at different time points. Here we suggest the use of the distributions of genomic distances between cfDNA fragments, which reflect the distributions of distances between biomolecular complexes that protect DNA from nuclease digestion in the cells of origin. Unlike previous inventions which suggest using the distributions of distances between cfDNA fragments to infer gene activity or the types of the cells of origin of cfDNA, the present invention uses the distributions of distances between cfDNA fragments for direct comparison between different samples. This allows monitoring of patients by comparing their own cfDNA samples taken at different time points, without the need for any reference dataset.

In particular, determining disease-specific characteristics in the genomic distribution of cfDNA fragments would be of value in expanding the use of liquid biopsy assays into a clinical tool for a wide range of medical conditions, and so would be beneficial in early diagnostics, patient monitoring and stratification. In fact, assessment of the genomic distribution of cfDNA fragments of healthy and diseased subjects may identify disease-specific changes in nucleosome positioning or changes in the distribution of other nucleoprotein complexes protecting genomic DNA from digestion by nucleases. Such disease-specific changes may form part of a liquid biopsy assay, which may then be used to diagnose, monitor a treatment response, and/or stratify a patient or a healthy person.

Certain embodiments of the present invention may provide assays based on disease-specific changes in nucleosome positioning. Such assays may have value in developing liquid biopsy assays that are both cost-effective and sensitive, and so can be used as an effective clinical tool across a wide range of medical conditions. Aptly the present invention provides methods for determining the distribution of distances between biomolecular complexes that protect DNA from nuclease digestion.

In certain embodiments, the present invention relates to a method of determining a genome-wide distribution of genomic distances between DNA fragments protected from nuclease digestion, the method comprising:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a first subset of the cfDNA fragments, each cfDNA fragment aligning to a first chromosome;
(e) selecting a further subset of cfDNA fragments, which align to a second chromosome;
(f) calculating the distribution of frequencies of distances between cfDNA fragments of:
   (fi) the first subset within a pre-determined genomic distance range from each other to form a distribution of frequencies of cfDNA distances from the first chromosome, and
   (fii) the second subset within a pre-determined genomic distance range from each other to form a distribution of frequencies of cfDNA distances from the second chromosome;
(g) averaging the distribution of frequencies of cfDNA distances across the first and second chromosomes to create a distribution of frequencies of cfDNA distances across multiple chromosomes; and
(h) analysing the distribution of frequencies of cfDNA distances to detect periodic patterns and calculating at least one periodicity parameter.

In certain embodiments, the method further comprises using said distribution as a marker of a disease or healthy condition.

In certain embodiments, the method further comprises using said distribution or its parts of periodicity parameters derived from it as a marker of a disease or healthy condition.

In certain embodiments, the method further comprises:
(i) using the distribution of frequencies of cfDNA distances or periodicity parameters derived from it as a marker of a disease or healthy state to perform cfDNA sample classification.

In certain embodiments, the at least one periodicity parameter is selected from a period(s) of oscillation of the distribution of frequencies of cfDNA distances, and/or relative numbers of cfDNA fragments mapped to different types of genomic DNA sequence repeats.

In certain embodiments, the DNA fragments are protected from nuclease digestion by a nucleosome, other DNA-bound nucleoprotein complex or a sequence-dependent DNA structure.

In certain embodiments the method further comprises:
(i) selecting one or more further subsets of cfDNA fragments, each further subset of cfDNA fragments aligning to a corresponding further chromosome, wherein the distribution of frequencies of cfDNA distances across multiple chromosomes is created from all the selected subsets of cfDNA.

In certain embodiments the method further comprises:
(j) selecting one or more further subsets of cfDNA fragments, each further subset of cfDNA fragments aligning to a corresponding further chromosome, wherein the distribution of frequencies of cfDNA distances across multiple chromosomes is created from all the selected subsets of cfDNA.

In certain embodiments, the method further comprises:
(j) using the distribution of frequencies of cfDNA distances or periodicity parameters derived from it as a marker of a disease or healthy state to perform cfDNA sample classification.

In certain embodiments, the genome-wide period of oscillation of the distribution of frequencies of cfDNA distances is a nucleosome repeat length (NRL) value.

In certain embodiments, step (h) comprises performing Fourier transform, discrete Fourier transform, fast Fourier transform or equivalent methods that decompose the distribution of frequencies of cfDNA distances to determine one or several periods of oscillation of distributions of frequencies of cfDNA distances, the method comprising:
(a) calculating the distribution of distances between DNA fragments protected from nuclease digestion, or
(b) calculating the distribution of the probabilities that a given genomic location represents the center of a nucleosomes or is covered by a nucleosome (so called aggregate nucleosome profiles), around genomic features such as transcription start sites, transcription factor binding sites, transcription termination sites, nucleosome depleted regions or stably positioned nucleosomes;
(c) calculating the Fourier transform (FT), discrete Fourier transform (DFT) or fast Fourier transform (FFT) of one of the said distributions;
(d) determining the prevalent frequencies of the said Fourier transform or equivalent transformations based on the peaks of the corresponding distribution of the transformation amplitudes as a function of the corresponding transformation frequencies;
(e) determining the values of the nucleosome repeat length (NRL) and other periods of oscillation of the original distributions defined in steps (a-b) as the inverse value of the said frequencies defined in step (d).

In certain embodiments, step (h) comprises performing linear regression on values corresponding to the locations of the summits of the peaks of the frequency distributions of cfDNA distances, to calculate the genome-wide nucleosome repeat length value (NRL).

In certain embodiments, the present invention relates to a method of determining a genome-wide distribution of genomic distances between DNA fragments protected from nuclease digestion and using the said distribution as a marker of a disease or healthy condition, the method comprising:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a first subset of the cfDNA fragments, each cfDNA fragment aligning to a first chromosome;
(e) selecting a further subset of cfDNA fragments, which align to a second chromosome;
(f) calculating the distribution of frequencies of distances between cfDNA fragments of:
   (fi) the first subset within a pre-determined genomic distance range from each other to form a distribution of frequencies of cfDNA distances from the first chromosome, and
   (fii) the second subset within a pre-determined genomic distance range from each other to form a distribution of frequencies of cfDNA distances from the second chromosome;
(g) averaging the distribution of frequencies of cfDNA distances across the first and second chromosomes to create a distribution of frequencies of cfDNA distances across multiple chromosomes;
(h) analysing the distribution of frequencies of cfDNA distances to detect periodic patterns and calculating at least one periodicity parameter; and
(i) using the distribution of frequencies of cfDNA distances or periodicity parameters derived from it as a marker of a disease or healthy state to perform cfDNA sample classification.

In a further aspect of the present invention, there is provided a method of determining a chromosome-wide distribution of genomic distances between DNA fragments protected from nuclease digestion:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a subset of cfDNA fragments, each of which aligns to a first chromosome residing in a genomic region of interest;
(e) calculating the distribution of frequencies of distances between cfDNA fragments of the subset of cfDNA fragments within a pre-determined distance range from each other; and
(f) analysing the said distribution of frequencies of cfDNA distances to detect periodic patterns and calculating at least one periodicity parameter.

In a further aspect of the present invention, there is provided a method of determining a chromosome-wide distribution of genomic distances between DNA fragments protected from nuclease digestion, the method comprising:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a subset of cfDNA fragments, each of which aligns to a first chromosome residing in a genomic region of interest;
(e) calculating the distribution of frequencies of distances between cfDNA fragments of the subset of cfDNA fragments within a pre-determined distance range from each other; and
(f) analysing the said distribution of frequencies of cfDNA distances to detect periodic patterns and calculating at least one periodicity parameter.

In certain embodiments, the method further comprises using said distribution as a marker of a disease or healthy condition.

In certain embodiments, the method further comprises:
(g) using the distribution of frequencies of cfDNA distances or periodicity parameters derived from it as a marker of a disease or healthy state to perform cfDNA sample classification.

In certain embodiments, the at least one periodicity parameter is selected from a period(s) of oscillation of the distribution of frequencies of cfDNA fragments, and/or the relative numbers of cfDNA fragments mapped to different types of DNA sequence repeats.

In certain embodiments, the chromosome-wide period of oscillation of the distributions of frequencies of cfDNA distances is a nucleosome repeat length (NRL) value.

In a further aspect of the present invention, there is provided a method of determining a distribution of genomic distances between DNA fragments protected from nuclease digestion, the method comprising:
(a) providing a plurality of nucleic acid sequences, wherein the plurality of nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence, wherein each mapped nucleic acid sequence is a cfDNA fragment, to a genomic location;
(d) selecting a subset of cfDNA fragments, each of which aligns to a region of interest in a first chromosome,
(e) calculating the distribution of frequencies of distances between cfDNA fragments of the subset of cfDNA fragments within a pre-determined distance range from each other within the genomic regions of interest, to form a distribution of frequencies of cfDNA distances; and
(f) analysing the said distribution of frequencies of cfDNA distances to detect periodic patterns and calculating at least one periodicity parameter.

In certain embodiments, the method further comprises using said distribution as a marker of a disease or healthy condition.

In certain embodiments, the method further comprises:
(g) using the distribution of frequencies of cfDNA distances or periodicity parameters derived from it as a marker of a disease or healthy state to perform cfDNA sample classification.

In certain embodiments, the at least one periodicity parameters is selected from a period(s) of oscillation of the distribution of frequencies of cfDNA distances and/or the relative numbers of cfDNA fragments mapped to different types of DNA sequence repeats.

In certain embodiments, the region of interest is selected from a region or a plurality of regions such as DNA sequence repeats, a set of binding sites of a transcription factor, a gene promoter and a region of differential DNA methylation.

In certain embodiments, the period of oscillation of the distribution of frequencies of cfDNA distances within the genomic regions of interest is a nucleosome repeat length (NRL) value.

In certain embodiments, step (d) comprises selecting of the region of interest based on the locations of gene bodies, enhancers, insulators, other regulatory genomic elements, binding sites of transcription factors, centromeric regions, heterochromatin regions, telomeric regions, DNA sequence repeats such as ALU, LINE, SINE, alpha-satellite repeats, microsatellite repeats, other types of DNA sequence repats, different types of chromatin domains such as topologically associating domains (TADs), lamina associated domains (LADs) or other types of domains, and/or genomic regions with enriched binding of different chromatin proteins and/or RNAs and/or regions with low/high/condition-sensitive DNA methylation or another epigenetic modification.

In certain embodiments, the distance between cfDNA fragments is calculated based on:
(i) the distribution between genomic coordinates of the centers of cfDNA fragments; and/or
(ii) the distribution between genomic coordinates of the edges of cfDNA fragments.

In certain embodiments, the biomolecular complexes protecting DNA from nuclease digestion are nucleosomes.

In certain embodiments, the reference genome is a human genome. For example, the reference genome may be selected from GRCh37/hg19, T2T CHM13, GRCh38/hg38 or other human genome. In certain embodiments, the reference genome is an animal genome, or any other genome.

In certain embodiments, the method of the present invention comprises selecting the first and optionally further subsets of cfDNA fragments based on one or more of the following:
(i) a predetermined length range of cfDNA fragments;
(ii) inclusion of one or more locations where the number of such mapped fragments exceeds a set threshold, which depends on the sequencing coverage of a given sample and
(iii) exclusion of locations where the number of such mapped fragments exceeds a set threshold, which depends on the sequencing coverage of a given sample.

In certain embodiments, the predetermined length range of cfDNA fragments is between 10-300 base pairs (bp), and is optionally 100-200 bp.

In certain embodiments, the predetermined length range of cfDNA fragments is between 10-10000 base pairs (bp), and is optionally 100-200 bp or 10-300 bp.

In certain embodiments, step (f) comprises performing linear regression on the coordinates of the summits of the peaks of the frequency distributions of cfDNA distances to calculate the NRL value.

In a further aspect of the present invention, there is provided a method of determining nucleosome repeat length (NRL) based on the analysis of the distribution of sizes of cfDNA fragments in the range of sizes from 100 bp to 1,000,000 bp, which represent stretches of DNA that were part of one and more than one nucleosome in the cells of origin, the method comprising:
(a) ex vivo extraction of total cfDNA or fractions of cfDNA from body fluid samples, including molecular fractions larger than mono-nucleosomes;
(b) determining the sizes of mono-, di-, tri-nucleosome fractions, as well as higher-multiple cfDNA fractions, using:
   (i) DNA size-fractionation such as gel-electrophoresis, capillary gel electrophoresis and/or membrane-based fractionation, or
   (ii) long-read sequencing, such as single-molecule real-time sequencing or Nanopore sequencing and;
(c) performing linear regression based on the distribution of cfDNA fragment sizes to determine the nucleosome repeat length (NRL).

In a further aspect of the present invention, there is provided a method of determining nucleosome repeat length (NRL) and the distribution of inter-nucleosome distances based on the analysis of the distribution of sizes of cfDNA fragments in the range of sizes from 50 bp to 1,000,000 bp, optionally 100 bp to 1,000,000 bp, which represent stretches of DNA that were part of one and more than one nucleosome in the cells of origin, the method comprising:
(a) ex vivo extraction of total cfDNA or fractions of cfDNA from body fluid samples, including molecular fractions larger than mono-nucleosomes;
(b) determining the sizes of mono-, di-, tri-nucleosome fractions, as well as higher-multiple nucleosome fractions, using:
   (i) DNA size-fractionation such as gel-electrophoresis, capillary gel electrophoresis Sanger sequencing and/or membrane-based fractionation, or
   (ii) long-read sequencing, such as single-molecule real-time sequencing or Nanopore sequencing and;
(c) performing linear regression based on the distribution of cfDNA fragment sizes to determine the nucleosome repeat length (NRL); or
(d) determining the locations of peaks of the density of cfDNA fragment sizes, such as mono-, di-, tri- and tertra-nucleosome fractions, and using the cfDNA fragment sizes associated with these peaks to perform machine learning for classification of cfDNA samples.

In certain embodiments, the method further comprises using said distribution as a marker of a disease or healthy condition.

In certain embodiments, the method further comprises using said distribution of sizes of multiple-nucleosome fractions, or distances between nucleosomes, or NRL, or other nucleosome periodicity parameters derived from these as a marker of a disease or healthy condition.

In certain embodiments, the method further comprises:
(d) using the distribution of frequencies of cfDNA distances or periodicity parameters derived from it as a marker of a disease or healthy state to perform cfDNA sample classification.

In certain embodiments, the method further comprises:
(d) using the distribution of sizes of multiple-nucleosome fragments or frequencies of inter-nucleosome distances or periodicity parameters derived from these as a marker of a disease or healthy state to perform cfDNA sample classification.

In certain embodiments, the method further comprises:
(d) using the distribution of sizes of multiple-nucleosome fractions, or distances between nucleosomes, or NRL, or other nucleosome periodicity parameters derived from these as a marker of a disease or healthy state to perform cfDNA sample classification.

In a further aspect of the present invention, there is provided a method of determining a subject's disease state using genome-wide nucleosome spacing, the method comprising:
(a) determining a genome-wide NRL value for a subject in at least one timepoint, according to the methods described above; and
(b) comparing the determined value to at least one set of reference nucleosome repeat length values;
wherein a time-dependent change of the determined NRL value or a match to any corresponding reference NRL value indicates a presence or absence of a disease or a specific state of healthy functioning.

In a further aspect of the present invention, there is provided a method of determining a subject's disease state using genome-wide nucleosome spacing, the method comprising:
(a) determining genome-wide sizes of multiple-nucleosome fractions, or distances between nucleosomes, or NRL, or other nucleosome periodicity parameters derived from these for a subject in at least one timepoint, according to the methods described above; and
(b) comparing the determined value to at least one set of reference nucleosome repeat length values;
wherein a time-dependent change of the determined sizes of multiple-nucleosome fractions, or distances between nucleosomes, or NRL, or other nucleosome periodicity parameters derived from these or a match to any reference values of these parameters indicates a presence or absence of a disease or a specific state of healthy functioning.

In certain embodiments, the NRL is 199-204 bp for non-malignant B-cells and is between 193-198 bp for B-cells in chronic lymphocytic leukemia (CLL), optionally wherein CLL subtype unmutated IGHV gene in general characterized by smaller NRL value than CLL subtype with mutated IGHV gene.

In certain embodiments, the NRL of cfDNA in healthy people is approximately 190 bp and wherein the NRL of cfDNA obtained from a patient suffering from breast cancer is 170-172 bp in chromosome 21 and genomic loci enriched with alpha-satellite repeats.

In certain embodiments, the NRL of cfDNA in healthy people is approximately 190 bp and wherein the NRL of cfDNA obtained from a patient suffering from cancer is 169-173 bp in chromosome 21 and other chromosomes and genomic loci enriched with alpha-satellite repeats.

In certain embodiments, step (b) comprises comparing:
(i) NRL determined using the same experimental method for the same subject at different time point(s), e.g. to monitor disease progression or response to therapy for a subject;
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; and/or
(iv) NRL determined using the same experimental method for an age- and gender-matched cohort of healthy people.

In a further aspect of the present invention, there is provided a method of determining a subject's disease state using chromosome-wide nucleosome spacing, the method comprising:
(a) determining a chromosome-wide NRL value for a subject in at least one timepoint, e.g. based on the methods described above; and
(b) comparing the determined value to at least one set of reference NRL values, wherein the time-dependent change of the determined NRL value or a match to any specific reference NRL values may indicate a presence or absence of a disease or a specific state of healthy functioning.

In certain embodiments, step (b) comprises comparing one or more of the following:
(i) NRL determined using the same experimental method for the same subject at different time point(s), e.g. to monitor disease progression or response to therapy for a subject;
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; or
(iv) NRL determined using the same experimental method for an age- and gender-matched cohort of healthy people.

In certain embodiments:
a) NRL is approximately 204 bp for chromosome 19 in non-malignant B-cells and is around 196 bp for chronic lymphocytic leukemia; and/or
b) in cfDNA of healthy people, NRL in chromosome 21 is around 190 bp and around 172-172 bp in a subject suffering from breast cancer in some genomic loci.

In certain embodiments:
a) NRL is approximately 204 bp for chromosome 19 in non-malignant B-cells and is around 196 bp for chronic lymphocytic leukemia; and/or
b) in cfDNA of healthy people, NRL in chromosome 21 and other chromosomes enriched with alpha satellite repeats is around 190 bp and around 169-172 bp in a subject suffering from breast cancer in some genomic loci.

In a further aspect of the present invention there is provided a method of determining a subject's disease state using nucleosome spacing in genomic regions of interest, the method comprising:
(a) determining the NRL value in a region of interest in at least one timepoint, e.g. based on the methods described above; and
(b) comparing the determined NRL value to at least one set of reference NRL values, wherein the time-dependent change of the determined NRL value or a match to any specific reference NRL values may indicate a presence or absence of a disease or a specific state of healthy functioning.

In certain embodiments, step (b) comprises comparing one or more of the following:
(i) NRL determined using the same experimental method for the same subject at different time point(s), e.g. to monitor disease progression or response to therapy for a subject;
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; or
(iv) NRL determined using the same experimental method for an age- and gender-matched cohort of healthy people.

In certain embodiments:
(a) NRL is around 200 bp for CLL-specific differentially methylated regions (DMR) in non-malignant B-cells and around 193 bp for aggressive types of chronic lymphocytic leukemia; and/or
(b) in cfDNA of healthy people, NRL in regions enclosing L1 DNA sequence repeats is around 191 bp and in patients with breast cancer and colorectal cancer NRL is around 188 bp and in patients with liver cancer, optionally NRL in regions enclosing L1 DNA sequence repeats decreases to around 186 bp.

In a further aspect of the present invention there is provided a method for use in determining a subject's disease state using the calculation of the relative numbers of cfDNA fragments mapping to different types of DNA sequence repeats, the method comprising:
(a) providing a plurality of nucleic acid sequences, wherein the plurality of nucleic acid sequences are obtained from cell free DNA (cfDNA) present in a sample from a subject or from a database;
(b) aligning each of the plurality of nucleic acid sequences to a reference genome or portion thereof to obtain a plurality of mapped nucleic acid sequences;
(c) determining the number of cfDNA fragments aligning to at least one type of DNA sequence repeats;
(d) determining a relative frequency of the representation of different repeat subtypes/families in a given cfDNA sample by performing normalization of the number of cfDNA fragments aligning to each family of DNA sequence repeats per 10,000,000 mapped reads, or use another type of normalization that makes this frequency independent of the sequencing coverage of a sample;
(e) comparing the frequency distribution of DNA sequence repeat subtypes of a sample with such distributions in other samples or in a reference database to perform sample classification using:
   (i) a predefined linear model or
   (ii) a machine learning model based on the vector composed of the relative number of different families of DNA sequence repeats represented in a given sample.

In a further aspect of the present invention there is provided a method for use in determining a subject's disease state using the calculation of the relative numbers of cfDNA fragments mapping to different types of DNA sequence repeats, the method comprising:
(a) providing a plurality of nucleic acid sequences, wherein the plurality of nucleic acid sequences are obtained from cell free DNA (cfDNA) present in a sample from a subject or from a database;
(b) aligning each of the plurality of nucleic acid sequences to a reference genome or portion thereof to obtain a plurality of mapped nucleic acid sequences;
(c) determining the number of cfDNA fragments aligning to at least one type of DNA sequence repeats;
(d) determining a relative frequency of the representation of different repeat subtypes/families in a given cfDNA sample by performing normalization of the number of cfDNA fragments aligning to each family of DNA sequence repeats per 10,000,000 mapped reads, or use another type of normalization that takes into account the sequencing coverage of a sample;
(e) comparing the frequency distribution of DNA sequence repeat subtypes of a sample with such distributions in other samples or in a reference database to perform sample classification using:
   (i) a predefined linear model or
   (ii) a machine learning model based on the vector composed of the relative number of different families of DNA sequence repeats represented in a given sample.

In certain embodiments, the predefined linear model may be based on a single parameter such as the relative numbers of DNA fragments mapped to alpha-satellite repeats in a sample, or more than one parameter, such as the relative number of DNA fragments mapped to alpha-satellite repeats, ALU repeats and/or L1 repeats.

In a further aspect of the present invention, there is provided a method for use in determining a subject's disease state using machine learning techniques for the analysis of nucleosome spacing in genomic regions of interest, the method comprising:
(a) determining the distributions of frequencies of cfDNA distances based on any method described above;
(b) creating a machine learning model based on techniques such as linear regression, logistic regression, support vector machines (SVM), convolutional neural networks (CNN), or deep learning, wherein the frequency distribution of cfDNA distances is represented as a vector characterising each cfDNA sample;
(c) training the said machine learning model using the frequency distributions of cfDNA distances for one or more healthy and diseased conditions; and
(d) performing the classification of the frequency distribution of cfDNA distances of a given subject using the said machine learning model.

In a further aspect of the present invention, there is provided a method for use in determining a subject's disease state using machine learning techniques for the analysis of nucleosome spacing in genomic regions of interest, the method comprising:
(a) determining the distributions of frequencies of cfDNA distances based on any method described above;
(b) creating a machine learning model based on techniques such as linear regression, logistic regression, support vector machines (SVM), convolutional neural networks (CNN), or deep learning, wherein the distribution of cfDNA distances or a set of variables derived from it, such as the locations of some of the peaks of the said distribution, is represented as a vector characterising each cfDNA sample;
(c) training the said machine learning model using the frequency distributions of cfDNA distances or a set of variables derived from the said distribution of cfDNA distances for one or more healthy and diseased conditions; and
(d) performing the classification of a given subject using the distribution of cfDNA distances or a set of variables derived from the said distribution using the said machine learning model.

In certain embodiments, there is provided a method for use in determining a subject's disease state using Fourier transform (FT), discrete Fourier transform (DFT), fast Fourier transform (FFT) or other Fourier transform-based algorithms for the analysis of nucleosome spacing genome-wide or in genomic regions of interest, the method comprising:
(a) determining one or more pronounced frequencies of the Fourier transform-based transformation of the distribution of nucleosome spacing for a subject in at least one timepoint, e.g. based on any method described above;
(b) computing the corresponding NRL values as the values inverse to the said frequencies, and
(c) comparing the determined values of said NRLs to at least one set of reference NRL values;
wherein a time-dependent change of the Fourier transform-based transformation amplitudes associated with the said frequencies and NRL values indicates a presence or absence of a disease or a specific state of healthy functioning.

Aptly, NRL values with the largest peaks of Fourier-transform amplitude for cfDNA from healthy people are about 200 bp and about 182 bp, and Fourier transform-based NRL value for cfDNA from breast cancer patients is about 182 bp (lacking the NRL value around 200 bp in the case of cancer).

In certain embodiments, the sets of reference NRL values and frequency distributions of cfDNA distances are from:
(i) a healthy cohort;
(ii) a diseased cohort;
(iii) cohorts of people with different ages;
(iv) cohorts of people with different ethnicities;
(v) cohorts of people with different weight or body mass index (BMI);
(vi) cohorts of people with different lifestyle; and/or
(vii) cohorts of people with different diet.

In certain embodiments, the disease is cancer and/or the specific state of healthy functioning is characterised by person's age, BMI, lifestyle or diet.

In certain embodiments, the method is for identifying the nucleosome positioning, or positioning of other nucleoprotein complexes, protecting DNA from digestion by nucleases in the genome of a plurality of subjects. In certain embodiments, the subjects are human subjects.

### BRIEF DESCRIPTION OF DRAWINGS

Certain embodiments of the present invention will now be described hereinafter, by way of example only, with reference to the accompanying drawings in which:

FIG. 1A shows a diagram depicting that circulating cell-free DNA (cfDNA) in body fluids comes from processes such as apoptosis, necrosis or NETosis, where DNA nucleases cut genomic DNA preferentially between nucleosomes. In the healthy person, most cfDNA in blood plasma has been released from blood cells. In patients with a disease the fraction of cfDNA originating from the diseased cell types may increase (Shtumpf M., Piroeva K. V., Agrawal S. P., Jacob D. R., Teif V. B. (2022). NucPosDB: a database of nucleosome positioning in vivo and nucleosomics of cell-free DNA. Chromosoma).

FIG. 1B shows a diagram depicting that the nucleosome repeat length (NRL) is defined as the average distance between centers of neighbouring nucleosomes (Teif V. B. and Clarkson C. T. (2019) Nucleosome Positioning. In Encyclopedia of Bioinformatics and Computational Biology (Ed.: S. Ranganathan, M. Gribskov, K. Nakai, and C. Schönbach), vol. 2, pp. 308-317. Oxford: Academic Press).

FIG. 2A shows a graph illustrating the distribution of distances between centers of cfDNA fragments calculated with NucTools for cfDNA sample from a breast cancer patient with ductal carcinoma (GEO accession number GSM1833259, SRA accession number SRR2130033).

FIG. 2B shows a graph illustrating an average distribution of distances between centers of cfDNA fragments created by averaging chromosomes-wide dyad-dad distances calculated in FIG. 2A.

FIG. 3A shows a graph illustrating the definition of peak summits of the average genome-wide distribution of distances between centres of cfDNA fragments from FIG. 2B.

FIG. 3B shows a plot of the locations of the peak summits of the average genome-wide profile of the distribution of distances between centers of cfDNA fragments from FIG. 2B, which is used to perform linear regression. The slope of the linear fit line is equal to the nucleosome repeat length value.

FIG. 4A shows a graph illustrating the distribution of distances between centers of cfDNA fragments calculated with NucTools for cfDNA sample from a healthy control (GEO accession number GSM1833278, SRA accession number SRR2130052).

FIG. 4B shows a plot of the locations of the peak summits of the average genome-wide profile of the distribution of distances between centers of cfDNA fragments from FIG. 4A, which is used to perform linear regression. The slope of the linear fit line is equal to the nucleosome repeat length value.

FIG. 5 is a flowchart outlining a method of determining the genome-wide nucleosome repeat length value according to certain embodiments of the present invention.

FIG. 6 shows a graph illustrating the genome-wide nucleosome repeat length calculated based on cfDNA taken from four healthy controls and four breast cancer samples. The nucleosome repeat length is significantly decreased in the cfDNA from breast cancer patients compared on this graph (P=0.045, two-sample t-test).

FIG. 7 shows a graph illustrating the genome-wide nucleosome repeat length of the cfDNA from healthy people, IGHV-mutated chronic lymphocytic leukaemia patients (M-CLL), and IGHV-unmutated chronic lymphocytic leukaemia patients (U-CLL). The genome-wide nucleosome repeat length decreases from ˜200 bp in non-malignant B-cells from healthy people (NBCs) to ˜198 bp in M-CLL (P=0.0028) to ˜195 bp in U-CLL (P=4.1×10⁻⁵).

FIG. 8 shows a graph illustrating the nucleosome repeat length (NRL) of 25-, 75- and 100-year-old people. The nucleosome repeat length is significantly increased in cfDNA samples from people with ages of 100 years compared to people with ages of 25 years and 75 years (P=0.037 and 0.02 respectively, two-sample t-test). NRLs for each individual (open circles), group-average values (open squares), medians (horizontal lines) and variance intervals (filled bars).

FIG. 9 shows a flowchart outlining a method of determining the nucleosome repeat length value for a single chromosome according to certain embodiments of the present invention.

FIG. 10A shows a graph illustrating distribution of distances between centres of cfDNA fragments for chromosome 21 calculated based on cfDNA from a breast cancer patient.

FIG. 10B shows locations of the peak summits of the profile of the distribution of distances between centers of cfDNA fragments from FIG. 10A, which is used to perform linear regression. The slope of the linear fit line is equal to the nucleosome repeat length value.

FIG. 11A shows a graph illustrating a comparison between the profiles of the distribution of distances between centers of cfDNA fragments for chromosome 21, averaged separately across four samples from healthy people (black line and the standard error of averaging as the grey cloud) and four breast cancer patients (red line and the standard error of averaging as the light red cloud). Without being bound by theory, the difference between healthy and cancer profiles in this case is mainly due to the different abundance of the fraction of cfDNA fragments mapped to alpha-satellite repeats (which have about 171 bp periodicity).

FIG. 11B shows a graph illustrating a comparison between the numbers of normalised occurrences of cfDNA fragments mapped to an example locus of alpha-satellite repeats for four samples from healthy people and six samples from breast cancer patients used in FIG. 11A, as well as eight samples from pancreatic cancer patients. Rhomboid-shaped symbols correspond to individual cfDNA samples. The values are normalised per 10,000,000 mapped reads per sample. The difference between the number of cfDNA fragments mapped to alpha-satellite repeats in pancreatic cancer and breast cancer samples is statistically significant (two sample t-test, Welch correction, P=0.033).

FIG. 12 shows a graph illustrating the distribution of distances between centers of DNA fragments obtained with MNase-assisted histone H3 ChIP-seq in B-cells from healthy people, IGHV-mutated chronic lymphocytic leukaemia patients (M-CLL), and IGHV-unmutated chronic lymphocytic leukaemia patients (U-CLL). The nucleosome repeat length for chromosome 19 decreases from ˜204 bp in non-malignant B-cells from healthy people (NBCs) to ˜197 bp in M-CLL to ˜196 bp in U-CLL.

FIG. 13 shows a flowchart outlining a method of determining a nucleosome repeat length value inside selected genomic regions of interest according to certain embodiments of the present invention.

FIG. 14 shows a graph illustrating the calculation of the nucleosome repeat length inside regions undergoing differential DNA methylation in chronic lymphocytic leukaemia, based on DNA fragments obtained with MNase-assisted histone H3 ChIP-seq in B-cells from healthy people (NBC) (A), IGHV-mutated chronic lymphocytic leukaemia patients (M-CLL) (B), and IGHV-unmutated chronic lymphocytic leukaemia patients (U-CLL) (C). The nucleosome repeat length for these differentially methylated regions decreases from ˜200 bp in NBCs to ˜196 bp in M-CLL to ˜193 bp in U-CLL.

FIG. 15A shows a graph illustrating the distribution of distances between centers of cfDNA fragments calculated with NucTools inside genomic regions around L1 repeats, averaged for four healthy cfDNA samples (SRA accession numbers SRR2130050, SRR2130051, SRR2130052; 21229993).

FIG. 15B shows a plot of the locations of the peak summits of the profile of the distribution of distances between centers of cfDNA fragments from FIG. 15A, which is used to perform linear regression. The slope of the linear fit line is equal to the nucleosome repeat length value, NRL=190.7+/−1.1 bp.

FIG. 16A shows a graph illustrating the distribution of distances between centers of cfDNA fragments calculated with NucTools inside genomic regions around L1 repeat, for a sample from a patient with liver cancer (SRA accession number SRR2130016).

FIG. 16B shows a plot of the locations of the peak summits of the profile of the distribution of distances between centers of cfDNA fragments from FIG. 16A, which is used to perform linear regression. The slope of the linear fit line is equal to the nucleosome repeat length value, NRL=186.3+/−1.7 bp.

FIG. 17A shows a graph illustrating the distribution of distances between centers of cfDNA fragments calculated with NucTools inside genomic regions around L1 repeat, for a sample from a patient with liver cancer (SRA accession number SRR2130035).

FIG. 17B shows a plot of the locations of the peak summits of the profile of the distribution of distances between centers of cfDNA fragments from FIG. 17A, which is used to perform linear regression. The slope of the linear fit line is equal to the nucleosome repeat length value, NRL=184 +/−1.6 bp.

FIG. 18A shows a graph illustrating the nucleosome occupancy profile near binding sites of a chromatin protein CTCF in a breast cancer cell line MCF-7.

FIG. 18B shows a graph illustrating fast Fourier transform (FFT) of the nucleosome occupancy profile shown in FIG. 18A. Upper panel shows the FFT phase as a function of frequency. Lower panel shows the FFT amplitude as a function of frequency. The amplitude graph is used to determine the prevalent frequencies in a given sample. Two peaks can be observed, with the first peak at frequency 0.0056 determining NRL 178.6 bp (1/0.0056).

FIG. 19A shows a graph illustrating fast Fourier transform (FFT) of the profile of the distribution of distances between cfDNA fragments for a breast cancer cfDNA sample (SRA accession number SRR2130011). The amplitude versus frequency graph (bottom) defines the major frequency as 0.0054945, which translates to the NRL value 182 bp (1/0.0054945).

FIG. 19B shows a graph illustrating fast Fourier transform (FFT) of the profile of the distribution of distances between cfDNA fragments for a breast cancer cfDNA sample (SRA accession number SRR2130043). The amplitude versus frequency graph (bottom) defines the major frequency as 0.0054945, which translates to the NRL value 182 bp (1/0.0054945).

FIG. 20A shows a graph illustrating fast Fourier transform (FFT) of the profile of the distribution of distances between cfDNA fragments for a healthy cfDNA sample (SRA accession number SRR2130050). The amplitude versus frequency graph (bottom) defines more than one frequency. The first frequency is 0.004995, which translates to the NRL value 200.2 bp (1/0.004995) (blue arrow). A secondary frequency 0.0054945, which translates to the NRL value 182 bp (red arrow), has smaller amplitude in this healthy cfDNA sample.

FIG. 20B shows a graph illustrating fast Fourier transform (FFT) of the profile of the distribution of distances between cfDNA fragments for a healthy cfDNA sample (SRA accession number SRR2130051). The amplitude versus frequency graph (bottom) defines more than one frequency. The first frequency is 0.004995, which translates to the NRL value 200.2 bp (1/0.004995) (blue arrow). A secondary frequency 0.0054945 translates to the NRL value 182 bp (red arrow).

### DETAILED DESCRIPTION

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology, recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Système International de Unitese (SI) accepted form. Numeric ranges are inclusive of the numbers defining the range.

Aspects of the present invention provide a method to determine a distribution of distances between biomolecular complexes that protect DNA from nuclease digestion. Aptly the distribution may relate to nucleosome positioning or the genomic distribution of other nucleoprotein complexes protecting DNA from nuclease digestion. Aptly, the method may be used to define disease-specific changes in the distribution of distances between biomolecular complexes that protect DNA from nuclease digestion. In an embodiment, the method may be used to determine nucleosome positioning determined based on cfDNA, e.g. cfDNA extracted from a body fluid of a subject, such as blood plasma or urine. The subject may be a heathy subject or may be a patient suffering from or suspected of suffering from a disorder. Aptly, assessment of the disease-specific changes in nucleosome positioning may form part of a liquid biopsy assay, which may then be used to diagnose, monitor, and/or stratify a patient.

The term “subject” as used herein may refer to any animal, mammal, or human. In some embodiments, the subject is a human. In some embodiments, the subject may be a heathy subject. In some embodiments, the subject may be a subject suffering from or suspected of suffering from a condition or disorder. Details of potential condition and disorders, including pathological disorders, are provided herein. In some embodiments, the subject may be a subject who is in or suspected of being in remission from a disorder.

Aptly, the methods described herein may identify disease-specific changes in nucleosome positioning in a genomic region.

The term “genomic region” as used herein generally refers to any region of the genome (e.g., a range of base pair positions), e.g., the entire genome, chromosome, gene, exon, a set of binding sites of a transcription factor or a set of DNA sequence repeats. The genomic region may be a continuous or discontinuous region. A “locus” (or “locus”) can be part or all of a genomic region (e.g., part of a gene, or a single nucleotide of a gene). The genome may be a human genome.

As used herein the term “region of interest” is defined based on the locations of gene bodies, enhancers, insulators, other regulatory genomic elements, binding sites of transcription factors, centromeric regions, heterochromatin regions, telomeric regions, DNA sequence repeats such as ALU, LINE, SINE, alpha-satellite repeats, microsatellite repeats, other types of DNA sequence repeats, or genomic regions with enriched binding of different chromatin proteins or RNAs.

The methods and system of certain embodiments comprise the use of a “reference genome”. The term “reference genome” is used to refer to a nucleic acid sequence database that is assembled from genetic data and intended to represent the genome of a species. Aptly, the reference genome is haploid. Aptly, the reference genome does not represent the genome of a single individual of that species, but rather is a mosaic of several individual genomes.

A reference human genome may be hg19. The hg19 human genome is disclosed https:/www.ncbi.nlm.nih.gov/assembly/GCF_000001405.13/.

In alternative embodiments, the reference human genome is GRCh38. The GRCh38 human genome is disclosed https://www.ncbi.nim.nih.gov/assembly/GCF_000001405.39.

In alternative embodiments, the reference human genome is CHM13 (T2T-CHM13). The CHM13 (T2T-CHM13) human genome is disclosed https://www.ncbi.nlm.nih.gov/assembly/GCA_009914755.4/.

As used herein the term “liquid biopsy” refers to the sampling and analysis of non-solid biological tissue. This is a powerful diagnostic and monitoring tool and has the benefit of being largely non-invasive, and so can be carried out more frequently. Non-limiting examples of liquid biopsy' sources include blood plasma, cerebrospinal fluid, urine or other bodily fluids. Liquid biopsies may be collected and purified by any means known in the art, with the method of extraction likely to depend on the source of the biopsy and the desired application.

A wide variety of biomarkers may be sampled and studied from the collected liquid biopsy, to detect or monitor a range of diseases and/or conditions. Aptly, the type of biomarker sampled from the liquid biopsy is dependent on the condition being tested and/or diagnosed. For example if the condition is cancer, then circulating tumor cells (CTCs) and/or circulating tumor DNA (ctDNA) are collected, whereas if the condition is a myocardial infarction, circulating endothelial cells (CECs) are sampled.

As used herein the term “cell-free DNA” (“cfDNA”) refers to non-encapsulated DNA (deoxyribonucleic acid) in body fluids such as blood plasma, urine, eye humour and cerebrospinal fluid. These nucleic acid fragments are usually of varying size, with over-representation of sizes similar to the length of DNA wrapped around a histone octamer, as well as its multiples. A nucleosome is the combination of about 147 DNA base pairs wrapped around the histone octamer, which usually consists of the following histone subunits: (H2A-H2B)-(H3-H4)-(H3-H4)-(H2A-H2B). A 147 bp segment of DNA is wrapped around the histone octamer in 1.65 turns. Histone H1 (linker histone) may be also involved in nucleosome packing.

Although the mechanisms of cfDNA release are not entirely understood, it is known that cfDNA can enter the bloodstream (or other bodily fluids) as a result of apoptosis or necrosis, as well as active extraction of sections of nucleic acids from the cell (e.g. in NETosis). Elevated cfDNA levels correlate with all-causes mortality, and so cfDNA is generally considered a prognostic factor and a biomarker. Based on the characteristics and accessibility of cfDNA it is deemed a biomarker of growing interest, and a tool in diagnostics and therapy-efficiency monitoring.

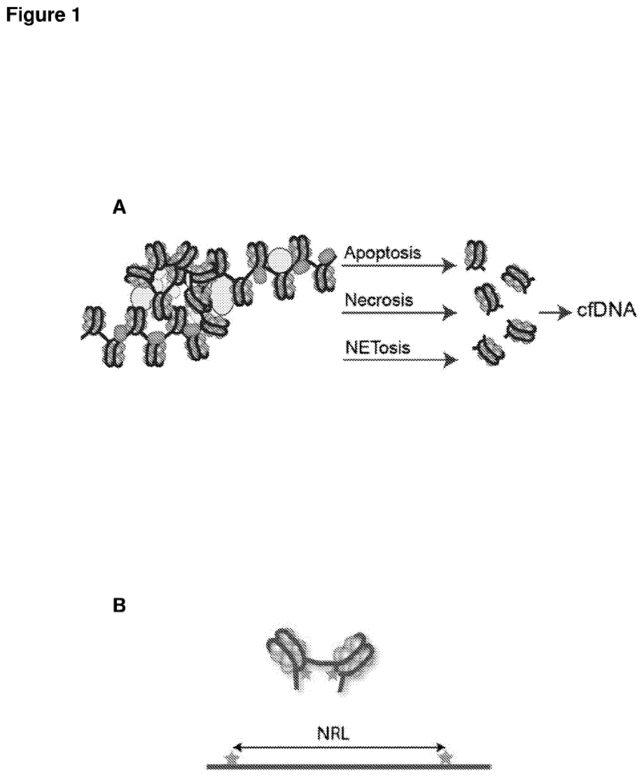

Aptly a liquid biopsy may comprise one or more sub-types of cfDNA including, but not limited to, circulating tumor DNA (ctDNA), circulating cell-free mitochondrial DNA (ccf mtDNA), and cell-free fetal DNA (cffDNA), as well as the total fraction of cell-free DNA (cfDNA). cfDNA may be collected and purified by any means known in the art, with the method of extraction likely to depend on source of liquid biopsy and the desired application. As shown in FIG. 1, cell-free DNA (cfDNA) in body fluids comes from processes such as apoptosis, necrosis or NETosis, where DNA nucleases cut genomic DNA preferentially between nucleosomes. In the healthy person, cfDNA in blood plasma can be released from blood cells as well as a smaller fraction from other cell types. In patients with a disease the fraction of cfDNA originating from the diseased cell types may increase. In healthy people the amount of cfDNA can differ depending on their age, diet, physical activity, stress, environmental conditions and other aspect of the life cycle.

Although cfDNA fragments detected in blood predominantly result from genomic regions protected from nuclease digestion by nucleosomes, some fragments result from genomic regions protected from nuclease digestion by molecular complexes other than nucleosomes. Such molecular complexes may include, but are not limited to, bound transcription factors, RNA polymerase and other nucleoprotein complexes.

The preference for DNA cutting by nucleases may also depend on the DNA sequence, and such preferences may be amplified in the case of DNA sequence repeats. Therefore, cfDNA fragments may also reflect the distribution of cfDNA fragments mapped to different genomic DNA sequence repeats. The fractions of cfDNA fragments mapped to such repeats may change in a disease (as shown below), and so can be used for diagnostics.

As used herein, the term “nucleosome-protected DNA fragments” relates to fragments of genomic DNA protected from nuclease digestion by the nucleosome. These can be defined either in the cells based on MNase-seq or similar chromatin digestion methods, or based on cell-free DNA (cfDNA), where digestion is done by apoptotic nucleases.

As used herein, “DNA fragments protected from nuclease digestion” may be protected from digestion by nucleosomes, some chromatin complexes other than conventional nucleosomes (e.g. incomplete nucleosomes such as hexasomes, transcription factors, RNA Pol II, etc), as well as by different properties of the DNA itself, which depends on the DNA nucleotide sequence.

As used herein the terms “biomolecular complexes that protect DNA from nuclease digestion” and “nucleoprotein complexes that protect DNA from nuclease digestion” refer to any RNA, protein or portion thereof that interacts with DNA to form a complex (via direct or indirect binding) and therefore prevents nuclease binding and subsequent digestion. Examples of such complexes include MeCP2 proteins, Xist RNA, transcription pre-initiation complex, enhanceosome and various chromatin remodellers. Aptly the genomic DNA protected by such complexes from nuclease digestion may become a fraction of cell free DNA. Aptly the protein may be a single subunit or in a complex e.g., a nucleosome.

In certain embodiments the present invention provides a method to determine a genome-wide distribution of distances between nucleosomes or other biomolecular complexes protecting DNA from nuclease digestion based on cell-free DNA (cfDNA). In certain embodiments, the method may determine the genomic-wide distribution of distances between nucleosomes or other biomolecular complexes protecting DNA from nuclease digestion based on cell-free DNA (cfDNA) obtained from a sample e.g., a liquid biopsy taken from a subject.

Certain embodiments of the present invention comprise sequencing one or more regions of a nucleic acid molecule. In certain embodiments, the nucleic acid molecule is a protein-associated DNA molecule e.g. a DNA molecule which is wrapped around a histone octamer.

In certain embodiments, information regarding the protein-wrapped DNA molecule is provided in a database e.g. a database comprising details of cell-free DNA from a plurality of subjects.

In certain embodiments, sequencing of protein-wrapped DNA e.g. cfDNA is based on published cfDNA datasets. An example of a database comprising cfDNA datasets is NucPosDB (https://generegulation.org/nucposdb/). NucPosDB also comprises nucleosome positioning maps in vivo (https://generegulation.org/nucposdb/).

In certain embodiments, the method comprises identifying nucleic acid molecules that are comprised in a sample comprising cfDNA. Optionally, the sample is obtained from a subject with a condition or disorder. The nucleic acid molecules may be processed to provide a plurality of reads. In one instance, these read-outs may include determining changes of nucleosome positioning. In one instance, changes in nucleosome positioning derived from cfDNA may be compared with nucleosome positioning in normal/disease tissues (e.g., tissues involved in a predefined condition), using methods such as MNase-seq, ATAC-seq, ChIP-seq, MNase-assisted histone H3 ChIP-seq, CUT&Tag, CUT&RUN or related.

MNase-seq (Micrococcal Nuclease digestion followed by deep sequencing) is a technique used to measure DNA protection by nucleosomes. The technique relies upon the non-specific endo-exonuclease micrococcal nuclease, an enzyme derived from Staphylococcus aureus to bind and cleave protein-unbound regions of DNA on chromatin. DNA bound to histones or other chromatin-bound proteins is preferentially protected from digestion. The uncut DNA is then purified and sequenced.

In certain embodiments, MNase-seq may be combined with or substituted by ChIP-seq, ATAC-seq, CUT&RUN and/or CUT&Tag sequencing. CUT&RUN sequencing, which is also known as cleavage under targets and release using nuclease, is a technique combining antibody-targeted controlled cleavage by micrococcal nuclease with massively parallel sequencing.

CUT&Tag sequencing (Cleavage under Targets and Tagmentation) is based on ChIP principles i.e. antibody-based binding of the target protein or histone modification of interest but instead of an immunoprecipitation step, antibody incubation is directly followed by shearing of the chromatin and library preparation.

In certain embodiments, the method comprises obtaining nucleic acid sequence information using an ATAC-seq (Assay for Transposase-Accessible Chromatin using sequencing) technique. ATAC-seq utilises hyperactive transposases to insert transposable markers with specific adapters, capable of binding primers for sequencing, into open regions of chromatin. Sequences adjacent to the inserted transposons can be amplified allowing for determination of accessibly chromatin regions.

In certain embodiments, the method comprises obtaining nucleic acid sequence information using a ChIP-seq (chromatin immunoprecipitation followed by sequencing) technique. Typically the ChIP method uses an antibody for a specific DNA-binding protein or a histone modification to identify enriched loci within a genome. ChIP-seq can be performed on live cells as well as on circulating nucleosomes or fragments of cfDNA bound to proteins while released to body fluids. ChIP-seq performed in cells usually includes a step of random cutting chromatin into pieces, either with the help of sonication or with the help of enzymes such as MNase. In the latter case, the method is referred to as MNase-assisted ChIP-seq. MNase-assisted histone H3 ChIP-seq employs an antibody against histone H3, which is present in most nucleosomes, and is one of the methods used to map genomic nucleosome locations.

Isolated cfDNA may be analysed by any means known in the art, non-limiting examples include 1^{st} generation sequencing techniques such as Maxam-Gilbert sequencing and Sanger sequencing; next generation sequencing techniques such as pyrosequencing (Roche 454); sequencing by ligation (SOLID); sequencing by synthesis (Illumina); IonTorrent/Ion Proton (ThermoFisher); long-read sequencing including SMRT sequencing (Pacific Biosciences) and Nanopore sequencing (Oxford Nanopore); polymerase chain reaction (PCR), PCR amplicon sequencing, hybrid capture sequencing, enzyme-linked immunosorbent assays (ELISA) and other methods. As a non-limiting example, cfDNA may be analysed by PCR to assess a specific nucleotide sequence, alternatively the cfDNA may be analysed by DNA sequencing methods to assess all the cfDNA present in the sample. Suitable DNA sequencing methods include, but are not limited to, PCR amplicon sequencing, hybrid capture sequencing, or any method known in the art. As a further non-limiting example, isolated cfDNA may be analysed by massively parallel sequencing (MPS). In particular, any appropriate method should aptly avoid contamination, especially in relation to ruptured blood cells.

Next-generation sequencing methods which may have utility in embodiments of the present invention include for example massive parallel sequencing. NGS platforms include, for example, Roche 454, Illumina NovaSeq, Illumina NextSeq, Illumina MiSeq, Illumina HiSeq, Illumina Genome Analyser IIX, Life Technologies SOLID, Pacific Biosciences SMRT, ThermoFisher IonTorrent/Ion Proton, Oxford Nanopore MinION, Oxford Nanopore GridION and Oxford Nanopore PromethION.

In certain embodiments, the methods and system are for determining a distribution of distances between biomolecular complexes that protect DNA from nuclease digestion.

As used herein the term nucleosome positioning refers to the location of nucleosomes with respect to the genomic DNA sequence. The nucleosome is the basic unit of eukaryotic chromatin, consisting of a histone core around which DNA is wrapped. Each nucleosome typically contains 147 base pairs (bp) of DNA, which is wrapped around the histone octamer.

The location of nucleosomes along the DNA and their chemical and compositional modifications are key to gene expression—and concomitant cell regulation. Thus, genomic nucleosome positions are non-random and reflect the unique biological processes of each cell. Compared to the slow changes reflected in DNA mutations or aberrant methylation—which may accumulate relatively slowly—genomic nucleosome positions provide almost real-time information on cell function and disease state. Thus, information on nucleosome positioning can provide a valuable diagnostic marker. Obtaining genome-wide nucleosome positioning maps based on tissues involved in disease, for example tumour tissues of cancer patients, may be an expensive and invasive procedure. On the other hand, inferring nucleosome positioning from cfDNA is less invasive.

Nucleosome positioning affects gene expression by modulating accessibility of transcription factors to their DNA binding sites as an important part of gene regulation in eukaryotes. Nucleosome maps thus provide insight into the regulatory mechanisms underlying disease mechanisms and can be potentially used for diagnostics. Therefore nucleosome positioning-centric analysis may reveal disease-specific mechanisms of epigenetic regulation and allow patient stratification more effectively than similar analyses with DNA accessibility, methylation or gene expression data. This suggests nucleosome positioning as important for understanding molecular mechanisms underlying disease progression and response to therapy.

Cell-free DNA of cancer patients is also known to be enriched with shorter fragments in comparison to healthy individuals. To separate this effect from nucleosome repositioning, nucleosome positioning analysis may apply filtering to analyze only DNA fragments of certain sizes, e.g. sizes between 100-200 bp. Without being bound by theory, cfDNA is generated by nucleases, which shred the chromatin of cells including cells undergoing apoptosis, necrosis or NETosis. These enzymes preferentially cut genomic DNA between nucleosomes. Therefore, nucleosome positioning is reflected in the cfDNA fragmentation patterns. Moreover, since the half-life of cfDNA in blood is in the range of several minutes, cfDNA extracted at any given time point represents a very recent snapshot of nucleosome positioning in the cells of origin.

Positioning and occupancy of nucleosomes are closely related concepts; nucleosome positioning is the distribution of individual nucleosomes along the DNA sequence and can be thought of in terms of a single reference point on the nucleosome, such as its center (dyad). Nucleosome occupancy, on the other hand, is a measure of the probability that a certain DNA region is wrapped onto a histone octamer.

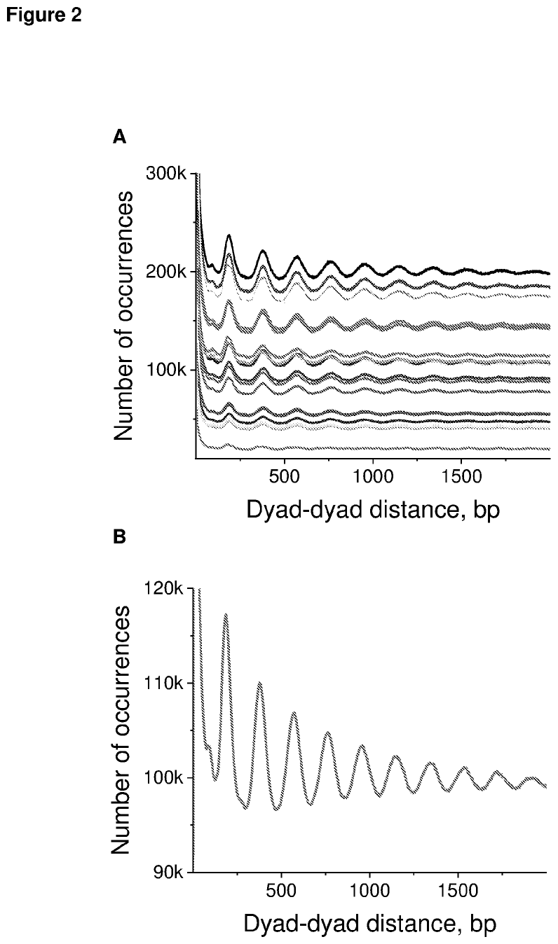

As used herein the “nucleosome repeat length” (NRL) refers to the average genomic distance between centers (dyads) of neighbouring nucleosomes. The nucleosome repeat length is equal to the average distance between the centers (dyads) of neighbouring nucleosomes along the DNA (FIG. 2), which can be defined either (1) locally at an individual genomic locus, or (2) across a number of different genomic loci of certain type, or (3) across a single chromosome, or (4) across the whole genome. Changes in nucleosome repeat length may account for significant changes of chromatin structure, for example the nucleosome repeat length difference between mouse embryonic stem cells and differentiated fibroblasts is ˜5 bp. NRL is an important physical chromatin property that determines its biological function and can be defined either as a genome-average value, or as an average for a smaller subset of genomic regions e.g., specific chromosomes or regions of interest.

Without being bound by theory, the term “nucleosome repeat length” defined above also refers, in the context of the current method, to the average genomic distance between centers of any neighbouring DNA-organising structures that lead to formation of regular genomic distances between digested DNA fragments in analogy to nucleosomes. Such DNA-organising structures include DNA-bound biomolecules, as well as regular structures formed by the DNA itself, for example as a result of the presence of DNA sequence repeats, T-loops, R-loops, G-quadruplexes, binding sites of CTCF proteins or regions of locally melted DNA double helix.

Non-limiting examples of sequence-dependent DNA structure, include but are not limited to, T-loops, R-loops, G-quadruplexes and DNA sequence repeats.

As used herein, the term “DNA sequence repeats” (also known as repetitive elements, repeating units or repeats) refers to patterns of nucleotides that occur in multiple copies throughout the genome. Further different types of DNA sequence repeats include, but are not limited to, alpha-satellite repeats, L1 repeats and ALU repeats.

As used herein, the term “relative numbers of cfDNA fragments mapped to different types of DNA sequence repeats” refers to an absolute number of cfDNA fragments mapped to a given type of DNA sequence repeats (e.g., repeat subtypes/families) which has been normalised, for example, per 10,000,000 of total mapped cfDNA reads.

Without being bound by theory, the distribution of frequencies of cfDNA distances represents some mathematical function which may have regular oscillations. Aptly, the major period of such oscillations usually corresponds to the nucleosome repeat length (NRL). However, sometimes more than one periodicity of nucleosome arrangement can be detected, and also there may be also other secondary oscillations which can be also valuable for the diagnostics. In certain embodiments the present invention determines a period of oscillation for the distributions of frequencies of cfDNA distances. In certain embodiments, the present invention determines one or more periods of oscillation for the distributions of frequencies of cfDNA distances. In certain embodiments, the present invention determines two or more periods of oscillation for the distributions of frequencies of cfDNA distances.

As used herein, the term “period(s) of oscillation of distributions of frequencies of cfDNA distances” refers to the period(s) of the wave-function(s) approximating the distribution of frequencies of cfDNA distances. In the simplest case, this refers to the periodicity of summits of clearly visible smooth peaks on the distribution of frequencies of cfDNA distances separated by roughly equal distances.

As used herein, the term “Fourier transform” or “Fourier transformation” refers to a function derived from a given function and representing it by a series of sinusoidal functions. Aptly this mathematical function decomposes a waveform (which is a function of space, time or some other variable) into the frequencies that constitute said waveform, thereby providing another way to represent the waveform. The Fourier transform calculation can be carried out using existing software, for example software Origin (originlab.com), including in the form of fast Fourier transform (FFT).

The methods of certain embodiments of the present invention comprise performing Fourier transformation, discrete Fourier transformation (DTF), fast Fourier transformation (FFT) or equivalent methods that decompose the distribution of frequencies of cfDNA distances to determine one or more periods of oscillation for the distribution of frequencies of cfDNA distances. Aptly a period of oscillation for the distributions of frequencies of cfDNA distances includes the nucleosome repeat length (NRL).

In certain embodiments, the method and system comprise determining nucleosome dyad-dyad distances in an individual sample and/or an average nucleosome dyad-dyad distance of a predetermined cohort of subjects. For example, certain embodiments comprise determining an average nucleosome dyad-dyad distance of a set of subjects having the same condition.

The term “distribution of nucleosome-nucleosome distances” is equivalent to the term “distribution of nucleosome dyad-dyad distances” or “distribution of dyad-dyad distances” (and is sometimes also called “phasogramm”). Such distribution shows the histogram of frequencies or absolute numbers of occurrences for each nucleosome-nucleosome distance, usually within a window enclosing several nucleosomes.

In certain embodiments of the present invention the method of determining nucleosome dyad-dyad distances comprises selecting a subset of nuclease-protected DNA fragments (e.g. include only certain fragment sizes, and exclude locations where the number of such mapped fragments exceeds a set threshold).

In certain embodiments filtering parameters for selecting a subset of nuclease-protected DNA fragment include fragment size. Aptly the certain fragment size may range between around 100 to 200 bp, 110 to 190 bp or 120 to 180 bp. In some embodiments, the subset of nuclease-protected DNA fragments is between 120-180 bp.

As used herein and as described above the term “disease-specific changes in nucleosome positioning” refers to nucleosome positioning changes characteristic to a given disease; such changes being an analytical characteristic that can also inform about the severity of condition. Thus, not only can such disease-specific changes in nucleosome positioning be used to distinguish between healthy and non-healthy subjects, but also between different medical conditions, different levels of severity of the same medical condition, and different conditions of a healthy person. Aptly disease-specific changes in nucleosome positioning may be defined as changes of nucleosome repeat length or more generally as changes of the distribution of distances between cfDNA fragments.

In certain embodiments, a subject with a disease comprises a significantly different nucleosome repeat length as compared to the corresponding nucleosome repeat length in a normal subject i.e., a subject who is not suffering from a pathological disorder.

In certain embodiments, the nucleosome repeat length may comprise a genome-wide nucleosome repeat length. Alternatively, the nucleosome repeat length may comprise a nucleosome repeat length of a specific chromosome. Alternatively, the nucleosome repeat length may comprise a nucleosome repeat length of a subset of genomic regions of interest or and individual genomic locus.

Certain embodiments of the present invention provide a method of selecting disease-specific changes in nucleosome positioning. Aptly the disease-specific changes are inferred based on cfDNA.

Aptly the disease-specific changes in nucleosome positioning are capable of distinguishing between different medical conditions, including but not limited to, different types of cancer and systemic inflammation, as well as the problem of determining biological age in healthy individuals. Consequently, the applicability of disease-specific changes in nucleosome positioning as part of liquid biopsy clinical tools is general.

In certain embodiments, the method and systems comprise determining regions of a genome which are substantially the same within a subject class e.g. subjects which each have a condition. In certain embodiments, the method comprises obtaining a read from a cfDNA sample e.g. a cfDNA sample comprised in a dataset.

The term “read” refers to a sequence read from a portion of a nucleic acid sample. Typically, though not necessarily, a read represents a short sequence of contiguous base pairs in the sample. The read may be represented symbolically by the base pair sequence (in ATCG) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 10 bp) that can be used to identify a larger sequence or region, e.g. that can be aligned to the reference genome and specifically assigned to a chromosome or an extra-chromosomal location inside the cell.

In certain embodiments, the method comprises the use of threshold values. As used herein the term “threshold” refers to a predetermined number used in an operation. For example, a threshold value can refer to a value above or below which a particular classification applies.

In certain embodiments, the disease may be a cancer. In certain embodiments, the disease is a subtype of a cancer. In certain embodiments, the subject has a malignant tumour.

The cancer type may be selected from the group consisting of: solid tumours such as melanoma, skin cancers, small cell lung cancer, non-small cell lung cancer, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, endometrial cancer, kidney cancer, renal cell carcinoma, colon cancer, colorectal, pancreatic cancer, oesophageal carcinoma, brain/CNS cancers, head and neck cancers, neuronal cancers, mesothelioma, sarcomas, biliary (cholangiocarcinoma), small bowel adenocarcinoma, paediatric malignancies, epidermoid carcinoma, sarcomas, cancer of the pleural/peritoneal membranes and leukaemia, including acute myeloid leukaemia, acute lymphoblastic leukaemia, IGHV-mutated chronic lymphocytic leukaemia, IGHV-unmutated chronic lymphocytic leukaemia, multiple myeloma, as well as other cancer types.

In certain embodiments, the disease may be a neoplastic disease, for example, melanoma, skin cancer, small cell lung cancer, non-small cell lung cancer, salivary gland, glioma, hepatocellular (liver) carcinoma, gallbladder cancer, thyroid tumour, bone cancer, gastric (stomach) cancer, prostate cancer, breast cancer, ovarian cancer, cervical cancer, uterine cancer, vulval cancer, endometrial cancer, testicular cancer, bladder cancer, lung cancer, glioblastoma, thyroid cancer, endometrial cancer, kidney cancer, colon cancer, colorectal cancer, pancreatic cancer, oesophageal carcinoma, brain/CNS cancers, neuronal cancers, head and neck cancers, mesothelioma, sarcomas, biliary (cholangiocarcinoma), small bowel adenocarcinoma, paediatric malignancies, epidermoid carcinoma, sarcomas, cancer of the pleural/peritoneal membranes and leukaemia, including acute myeloid leukaemia, acute lymphoblastic leukaemia, and multiple myeloma. Treatable chronic viral infections include HIV, hepatitis B virus (HBV), and hepatitis C virus (HCV) in humans, simian immunodeficiency virus (SIV) in monkeys, and lymphocytic choriomeningitis virus (LCMV) in mice.

In certain embodiments, the disease may comprise disease-related cell invasion and/or proliferation. Disease-related cell invasion and/or proliferation may be any abnormal, undesirable or pathological cell invasion and/or proliferation, for example tumour-related cell invasion and/or proliferation.

In one embodiment, the neoplastic disease is a solid tumour selected from any one of the following carcinomas of the breast, colon, colorectal, prostate, stomach, gastric, ovary, oesophagus, pancreas, gallbladder, non-small cell lung cancer, thyroid, endometrium, head and neck, renal, renal cell carcinoma, bladder and gliomas.

In certain embodiments, the disease may comprise a subtype of a disease. For example in certain embodiments, the disease may be a subtype of a cancer. By way of example only, the disease may be a biomarker-positive cancer e.g. HER2+ breast cancer, or alternatively may be a biomarker-negative cancer e.g. HER2 negative breast cancer.

As used herein the term “IGHV-mutated” refers to immunoglobulin heavy chain gene (lgHV) mutation status. Without being bound by theory this status correlates with the clinical outcome of patients with chronic lymphocytic leukemia (CLL). The survival rate of patients with unmutated IgHV is usually worse than that of patients with mutated IgHV. In certain embodiments the cancer is IGHV-mutated chronic lymphocytic leukaemia. In certain embodiments the cancer is IGHV-unmutated chronic lymphocytic leukaemia.

In certain embodiments, the disease is an inflammatory disorder. The inflammatory disorder may be selected from lupus, asthma, rheumatoid arthritis, ulcerative colitis, Crohn's disease, myocarditis, pericarditis, multiple sclerosis, sepsis, psoriasis and the like.

In certain embodiments, the disease is an autoimmune disorder.

In certain embodiments, the diseased subject has a pathological disorder and the healthy subject has an absence of a pathological disorder.

As used herein, the term “healthy” refers to person in a good physical or mental condition not displaying clinical signs of disease, infection or illness.

In some embodiments, the method comprises comparing the subject with the disease or the healthy subject with a reference subject. In certain embodiments, the reference subject is healthy. In some embodiments, the reference subject has a disease or disorder, optionally selected from the group consisting of: cancer, normal pregnancy, a complication of pregnancy (e.g., aneuploid pregnancy), myocardial infarction, inflammatory bowel disease, systemic autoimmune disease, localized autoimmune disease, allotransplantation with rejection, allotransplantation without rejection, stroke, and localized tissue damage.

In certain embodiments, the present invention provides a method of determining a subject's nucleosome positioning based on cfDNA. Aptly nucleosome positioning may relate to genome-wide nucleosome positioning. Aptly nucleosome positioning may relate to chromosome-wide nucleosome positioning, for example, the nucleosome positioning of chromosome 21. In certain embodiments of the present invention, nucleosome positioning is defined by dyad-dyad distances between neighbouring nucleosomes. Aptly the average dyad-dyad distance between neighbouring nucleosomes is measured as the nucleosome repeat length.

In certain embodiments, the nucleosome repeat length in a normal, healthy, subject is between around 50 bp to around 300 bp. Aptly the nucleosome repeat length value is typically within the range listed above, typically determined with a precision around 0.1 bp.

In certain embodiments the genome-wide nucleosome repeat length in a normal, healthy, subject is between around 50 bp to around 300 bp. Aptly the genome-wide nucleosome repeat length value is typically within the range listed above, typically determined with a precision around 0.1 bp.

In certain embodiments the nucleosome repeat length for chromosome 21 in a normal, healthy, subject is between around 50 bp to around 300 bp. Aptly the nucleosome repeat length value for chromosome 21 is typically within the range listed above, typically determined with a precision around 0.1 bp. The distribution of cfDNA distances for chromosome 21 may include a periodicity around 170-172 bp associated with alpha-satellite repeats.

In certain embodiments the nucleosome repeat length for chromosome 7 in a normal, healthy, subject is between around 50 bp to around 300 bp. Aptly the nucleosome repeat length value for chromosome 7 is typically within the range listed above, typically determined with a precision around 0.1 bp. The distribution of cfDNA distances for chromosome 7 may include a periodicity around 170-172 bp associated with alpha-satellite repeats.

In certain embodiments the nucleosome repeat length for chromosome 19 in a normal, healthy, subject is between around 50 bp to around 300 bp. Aptly the nucleosome repeat length value for chromosome 19 is typically within the range listed above, typically determined with a precision around 0.1 bp. The distribution of cfDNA distances for chromosome 19 may include a periodicity around 204 bp.

In certain embodiments the nucleosome repeat length in a subject suffering from or suspected of suffering from a disorder e.g. a disease is between around 50 bp to around 300 bp. Aptly the nucleosome repeat length value is typically within the range listed above, typically determined with a precision around 0.1 bp.

In certain embodiments the genome-wide nucleosome repeat length in a diseased subject is between around 50 bp to around 300 bp. Aptly the genome-wide nucleosome repeat length value is typically within the range listed above, typically determined with a precision around 0.1 bp.

In certain embodiments the nucleosome repeat length for chromosome 21 in a diseased subject is between around 50 bp to around 300 bp. Aptly the nucleosome repeat length value for chromosome 21 is typically within the ranges listed above, typically determined with a precision around 0.1 bp. In the case of breast cancer, the distribution of cfDNA distances for chromosome 21 may include a periodicity around 170-172 bp associated with alpha-satellite repeats. The fraction of cfDNA fragments with such periodicity may reflect the cancer type and aggressiveness, which can be used for patient diagnostics, stratification and monitoring.

In certain embodiments the nucleosome repeat length for parts of chromosome 7 in a diseased subject is between around 5 bp to around 300 bp. Aptly the nucleosome repeat length value for chromosome 7 is typically within the ranges listed above, typically determined with a precision around 0.1 bp. In the case of breast cancer, the distribution of cfDNA distances for parts of chromosome 7 may include a periodicity with the period around 170-171 bp. The fraction of cfDNA fragments with such periodicity may reflect the cancer type and aggressiveness, which can be used for patient diagnostics, stratification and monitoring.

In certain embodiments the nucleosome repeat length for chromosome 19 in a diseased subject is between around 50 bp to around 300 bp. Aptly the nucleosome repeat length value for chromosome 19 is typically within the ranges listed above, typically determined with a precision around 0.1 bp. In the case of CLL, the distribution of cfDNA distances for chromosome 19 may include a periodicity around 196 bp. The fraction of cfDNA fragments with such periodicity may reflect the cancer type and aggressiveness, which can be used for patient diagnostics, stratification and monitoring.

The present disclosure also provides methods of diagnosing a disease or disorder based on the distances between biomolecular complexes that protect DNA from nuclease digestion. Aptly this is measured as a period(s) of oscillation of distributions of frequencies of cfDNA distances. Aptly the distance is a dyad-dyad distance between neighbouring nucleosomes, determined by the method according to the present invention and as disclosed herein.

In certain embodiments, the methods for determining the distribution of genomic distances between cfDNA fragments as detailed herein are then used for comparison of nucleosome positioning between samples, which can be done with a number of computational approaches.

In certain embodiments, the method comprises of the use of machine learning techniques such as linear regression, logistic regression, support vector machines (SVM), convolutional neural networks (CNN), deep learning or explainable artificial intelligence. In certain embodiments the method comprises the use of dimensionality reduction techniques, such as principal component analysis (PCA) t-distributed stochastic neighbour embedding (tSNE), k-means clustering, or unsupervised clustering.

In certain embodiments, the method comprises obtaining a sample comprising cell-free DNA from a subject suspected of having or having a disease.

Thus, in certain embodiments, the method comprises use of Principal Component Analysis (PCA). As used herein principal component analysis (PCA) is a technique for reducing the dimensionality of datasets. In order to interpret large datasets, methods are required that drastically reduce the dataset's dimensionality in an interpretable manner, while also preserving the information in the data. PCA is an adaptive descriptive data analysis tool, which creates new uncorrelated variables that successively maximize variance. This methodology reduces a dataset's dimensionality, thereby increasing interpretability but at the same time minimizing information loss. Furthermore, PCA can be effectively tailored to various data types and structures, hence can be used in numerous situations and disciplines.

In certain embodiments, the method comprises performing linear regression on the locations of the summits of two or more peaks of the distribution of distances between cfDNA fragments to calculate the nucleosome repeat length value. Aptly the nucleosome repeat length value is calculated by performing linear regression on the locations of the summits of two or more peaks of the distribution of distances between cfDNA fragments.

In certain embodiments, the method comprises performing linear regression on the locations of the summits of two or more peaks of the distribution of distances between cfDNA fragments to calculate the genome-wide nucleosome repeat length value.

In certain embodiments, the method comprises performing linear regression on the locations of the summits of two or more peaks of the distribution of distances between cfDNA fragments to calculate the chromosome-wide nucleosome repeat length value.

In certain embodiments the method comprises calculating the nucleosome repeat length value based on a single peak of the dyad-dyad distances profile.

In certain embodiments, the method comprises performing one or more analysis of the distribution of distances between cfDNA fragments e.g. Fourier Transformation/classification/clustering/machine learning/deep learning analysis.

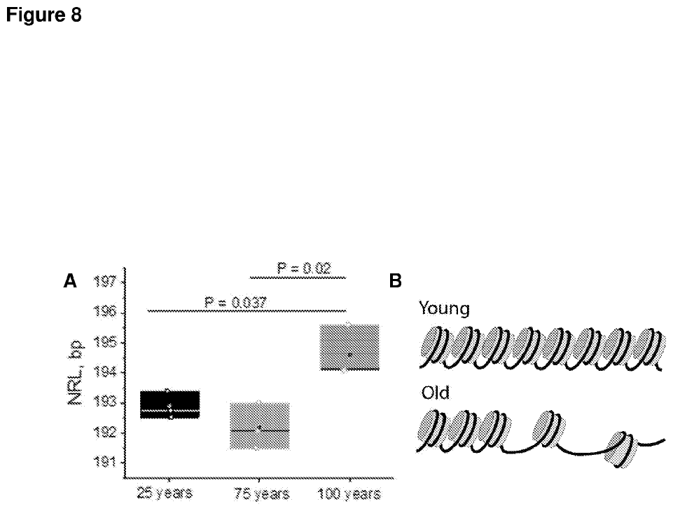

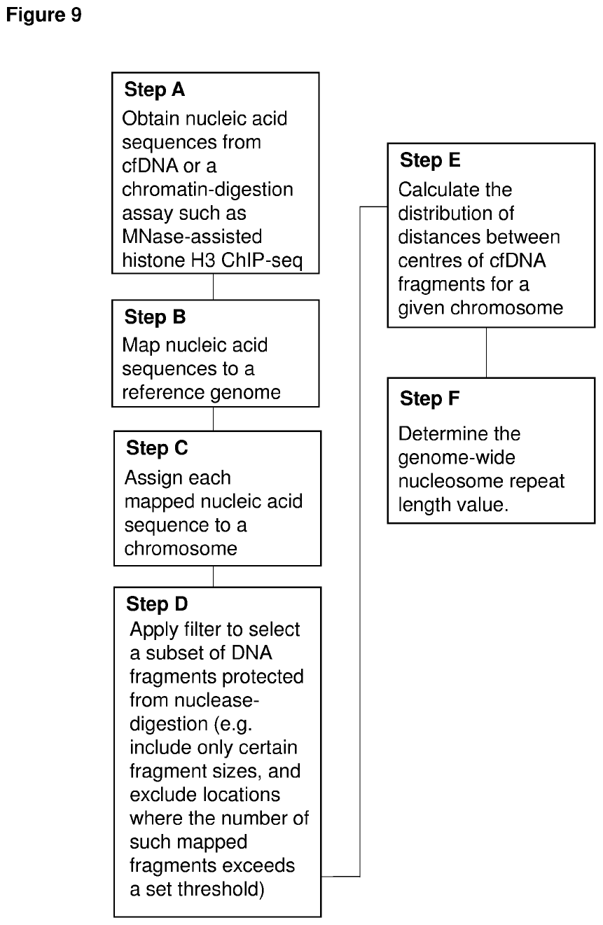

In certain embodiments, the method comprises inclusion or exclusion of one or more co-morbidities. Particularly, in certain embodiments, the method allows fine-tuning disease-specific nucleosome repeat length values to include/exclude the effect of different comorbidities. For example, cancer patients of different ages often have different cfDNA patterns. It is important to distinguish healthy ageing from different medical conditions. Aptly it has been identified that 100-year-olds display longer nucleosome repeat lengths compared to people with ages of 25 years and 75 years (FIG. 8). A set of age-sensitive nucleosome repeat length values that can be used for the estimation of the patient's age based on cfDNA can be compiled. Cancer patients of different ages have both cancer-specific nucleosome repeat length changes and age-specific nucleosome repeat length changes, therefore certain embodiments of the present invention take age-specific changes into account. This is exemplified by the reference nucleosome repeat length value sets, which are age-specific and so nullify age-specific cfDNA changes allowing analysis of only cancer-specific cfDNA changes. Similarly, the method of certain embodiments allows excluding other comorbidities-sensitive regions from sets of regions used in cfDNA-based medical diagnostics.

FIG. 8 shows a graph illustrating the nucleosome repeat length (NRL) of 25-, 75- and 100-year-old. The nucleosome repeat length is significantly increased in cfDNA samples from people with ages of 100 years compared to people with ages of 25 years and 75 years (P=0.037 and 0.02 respectively, two-sample t-test). NRLs for each individual (open circles), group-average values (open squares), medians (horizontal lines) and variance intervals (filled bars).

In certain embodiments, disease-specific changes of nucleosome positioning may include for example disease-specific changes of the average profiles of the distribution of distances between cfDNA fragments.

In certain embodiments of the present invention, a method of diagnosing a disease may comprise comparing a subject's nucleosome repeat length value with the reference nucleosome repeat length values.

Aptly the method comprises an automated procedure which processes all available datasets to calculate nucleosome-nucleosome distance distributions for discrete comparison of NRL values in diagnostic regions.

As used herein the term “machine learning” refers to an application of computational algorithms that provide the ability to automatically learn and improve from experience without being explicitly programmed. Machine learning focuses on the development of computer programs that can access data and through the use of statistical methods, algorithms are trained to make classifications or predictions, uncovering key insights within data mining projects. These insights subsequently drive decision making within applications.

In certain embodiments of the present invention, a method of diagnosing a disease may comprise the application of machine learning for multi-classification of the whole distribution of distances between cfDNA fragments rather than a single nucleosome repeat length value, this method would involve using a training set of distribution of distances between cfDNA fragments from a range of healthy and diseases conditions.

Aptly the method comprises an automated procedure which processes some available datasets to calculate the distribution of distances between cfDNA fragments for machine learning using the distributions of distances between cfDNA fragments per se.

In certain embodiments of the present invention, a system is provided which is configured to perform the methods of the invention. Aptly, the system is a computer-implemented system. The computer system can control various aspects of the disclosed method. The computer system may include a central processing unit (CPU), also referred to as a processor or computer processor. In certain embodiments, the processor may be a plurality of processors. The computer system may communicate with a memory or memory location. The computer system may comprise a computer or a mobile computer device e.g. a smartphone or a tablet. Also included in the computer system may be an electronic storage unit and one or more other systems. In certain embodiments, the computer system may comprise of a high-throughput computer cluster.

Without being bound by theory, the skilled person would readily be able to obtain the necessary raw data sequence reads for the presently disclosed methods and systems. Aptly the skilled person may obtain the raw sequence data from publicly available database (e.g., European Genome-phenome Archive (EGA), Short Read Archive (SRA), NucPosDB, Gene Expression Omnibus (GEO), the database of Genotypes and Phenotypes (dbGaP) and China National GeneBank DataBase (CNGBdb)). Aptly the skilled person may obtain the raw sequence data by sequencing cfDNA present in a sample obtained from a subject.

The methods and systems of the present disclosure can be implemented by one or more algorithms. The algorithm can be implemented by software when executed by a processor. In certain embodiments, determining a distribution of distances between biomolecular complexes that protect DNA from nuclease digestion may comprise the use of software packages, NucTools (https://generegulation.org/nuctools), BEDTools (https://bedtools.readthedocs.io/en/latest/), Bowtie or Bowtie2 (http://bowtie-bio.sourceforge.net/index.shtml), as well as other general-purpose bioinformatics tools for next generation sequencing analysis and custom-made scripts.

NucTools is also described in
Vainshtein, Y., Rippe, K. & Teif, V. B. “NucTools: analysis of chromatin feature occupancy profiles from high-throughput sequencing data.” BMC Genomics 18, 158 (2017).
https://doi.org/10.1186/s12864-017-3580-2.

BEDTools is also described in
Quinlan A R, Hall I M. 2010. “BEDTools: a flexible suite of utilities for comparing genomic features.” Bioinformatics 26:841-842.
https://doi.org/10.1093/bioinformatics/btq033

Bowtie is also described in
Langmead B, Trapnell C, Pop M, Salzberg S L. “Ultrafast and memory-efficient alignment of short DNA sequences to the human genome.” Genome Biol 10: R25.
https://doi.org/10.1186/gb-2009-10-3-r25

In certain embodiments the nucleosome repeat length value is calculated by performing linear regression.

Without being bound by theory, cancer samples may have shorter nucleosome repeat length than normal samples. Without being bound by theory, samples from more aggressive (advanced, higher grade) cancers may have shorter nucleosome repeat length than samples from less aggressive (less advanced, lower grade) cancers. This effect opens several possibilities for diagnostics along with patient stratification and monitoring.

Without being bound by theory, the change of the nucleosome repeat length in cancer may be caused by the overrepresentation of DNA sequence repeats, such as alpha-satellite repeats, ALU repeats, L1 repeats, and others. The distributions of distances between cfDNA fragments reflects the change in the relative abundance of such fractions. Therefore, the analysis of the distributions of distances between cfDNA fragments described in the current invention allows the effects of the relative abundance of such cfDNA fractions to be used in the patient diagnostics, stratification and monitoring.

Without being bound by theory, the change of the nucleosome repeat length in cancer may be caused by the relative composition of the fractions of cfDNA fragments with longer and shorter sizes. The distributions of distances between cfDNA fragments reflects the change in the relative abundance of such fractions. Therefore, the analysis of the distributions of distances between cfDNA fragments described in the present invention allows the effects of the relative abundance of such cfDNA fractions to be used in the patient diagnostics, stratification and monitoring.

Without being bound by theory, the change of the nucleosome repeat length in cancer may be caused by the relative composition of the fractions of cfDNA fragments coming from genomic regions with disease-specific differential changes of DNA methylation. The distributions of distances between cfDNA fragments reflects the change in the relative abundance of such fractions. Therefore, the analysis of the distributions of distances between cfDNA fragments described in the current invention allows patient diagnostics, stratification and monitoring.

Without being bound by theory, the change of the nucleosome repeat length in cancer may be caused by the relative composition of the fractions of cfDNA fragments coming from genomic regions with disease-specific differential changes of the abundance of linker histone H1 variants. The distributions of distances between cfDNA fragments reflects the change in the relative abundance of such fractions. Therefore, the analysis of the distributions of distances between cfDNA fragments described in the current invention allows patient diagnostics, stratification and monitoring.

Existing methods based on the analysis of copy number variations, or more generally on the quantification of cfDNA occupancy/density in certain regions are based on the assumption that the whole genome is sequenced homogeneously, which is not the case. The method based on the analysis of distribution of nucleosome-nucleosome distances proposed here is advantageous as the integral parameter, nucleosome repeat length, is robustly defined on the genome-wide level and so is more robust against experimental artefacts due to large statistical power.

In certain embodiments the present invention relates to a method of determining NRL based on the analysis of sizes of cfDNA fragments representing multiples of nucleosomes. Aptly this method involves the ex vivo extraction of total cfDNA or fractions of cfDNA from body fluid samples, including molecular fractions larger than mono-nucleosomes. Aptly this method comprises experimental methods such as gel-electrophoresis, capillary gel electrophoresis, mass-spectroscopy or any other method allowing to distinguish fragment sizes and charges of cfDNA molecules. Aptly this method comprises determining the sizes of mono-, di-, tri-nucleosome fractions, as well as higher-multiple cfDNA fractions, using long-read sequencing, such as single-molecule real-time sequencing (SMRT, Pacific Biosciences) or Nanopore sequencing (Oxford Nanopore).

### EXAMPLES

In the following, the invention will be explained in more detail by means of non-limiting examples of specific embodiments.

### Example 1—Method to Determine Nucleosome Repeat Length

### Calculations Setup.

Calculations shown in the above Figures were performed using the University of Essex computer cluster, ceres.essex.ac.uk, through bash scripting and interactive command line using Linux, PUTTY terminal and WinSCP file manager.

Software packages NucTools [1], BedTools [Quinlan A R, Hall I M. 2010. “BEDTools: a flexible suite of utilities for comparing genomic features.” Bioinformatics 26:841-842] and Bowtie [Langmead B, Trapnell C, Pop M, Salzberg S L. “Ultrafast and memory-efficient alignment of short DNA sequences to the human genome.” Genome Biol 10: R25] and complementary R and Shell scripts included herein were used to perform data processing. OriginPro 2020 (originlab.com) was used for data visualisation and statistical analysis.

### Downloading Data.

Fastq files with raw reads from the aforementioned studies were obtained from the Short Read Archive (SRA) (accession numbers SRR2130050, SRR2130051, SRR2130052, 21229993, SRR2130016, SRR2130035, SRR2130020, SRR2130023, SRR2130024, SRR2130044, SRR2130046. SRR2130047, SRR2130048, SRR2130049, SRR2130045, SRR2130043, SRR2130033, SRR2130032, SRR2130011, SRR2130004, SRR999659, SRR999660 and SRR7170698-SRR7170709) using SRA Tools to download the files from SRA and split files into two as the original libraries are paired-end in both studies. The dataset of nucleosome positioning in non-malignant B-cells and CLL patients determined with MNase-assisted histone H3 ChIP-seq reported by Mall et al., 2019 (Mallm J. P., (2019) Linking aberrant chromatin features in chronic lymphocytic leukemia to transcription factor networks. Mol Syst Biol 15, e8339) is available in the European Genome-phenome Archive (EGA) under accession number EGAS00001002518. The processed version of this dataset, mapped with Bowtie as detailed below, has been also deposited to the Gene Expression Omnibus (GEO) under accession number GSE158745.

### Reads Alignment and Pre-Processing.

The sequencing reads were mapped to the hg19 human reference genome using Bowtie [4] with parameters set for paired-end reads, allowing up to 2 mismatches, only considering uniquely mappable reads, suppressing all alignments for a read if more than 1 reportable alignments exist for it. The following pre-processing was performed with NucTools. The output Bowtie .map files were converted to BED format using bowtie2bed.pl script (part of NucTools package), and the paired-end reads were combined into one line, adding the fragment length as a new column using NucTools script extend_PE_reads.pl. The mapped.bed files were split into individual chromosomes using NucTools script “extract_chr_bed.pl”.

### Calculation of Nucleosome Repeat Length (NRL) Using the Linear Regression Method.

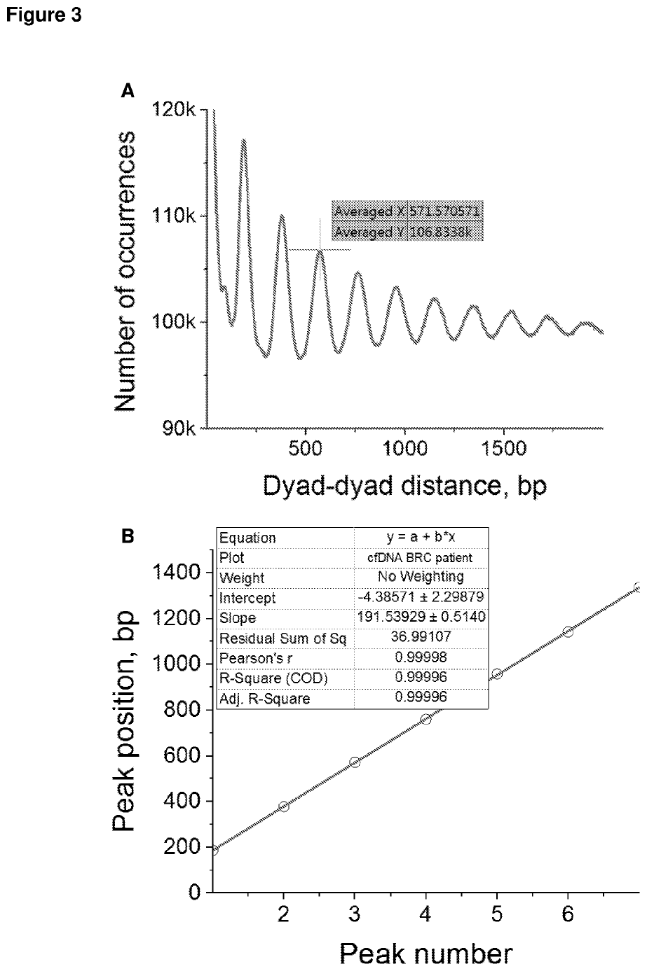

1. Select a fraction of cfDNA fragments of interest. For example, only DNA fragments with sizes 120-180 bp were considered in the calculations of the above FIGS. 2-4, 6-8, 10-12 and 14-17.
2. Calculate for each sample the histogram of nucleosome dyad-dyad distances using NucTools as follows:
   split the BED file with all mapped paired-end reads into individual chromosomes and run the calculation in a loop, separately for each chromosome (autosomal chromosomes plus chromosomes X and Y)
   In the calculations reported in FIGS. 2-4, 6-8, 10-12 and 14-17, NucTools script nucleosome_repeat_length.pl was run with parameters --delta=2000 -- MaxNr=10000000 --apply_filter
   Parameter --delta=2000 sets the maximum distance from start of the reference nucleosome (DNA fragment) to the last one in the calculations
   Parameter --MaxNr=10000000 sets the maximum number of such reference nucleosomes considered per chromosome.
   The use of parameter “--apply_filter” means that DNA fragments with piles more than 20 are removed from the analysis (genomic locations which have more than 20 DNA fragments centered at them).
3. Determine positions of the summits of peaks on the histograms and use these for linear regression to calculate NRL, as follows:
   create a graph with the distributions of genomic distances between DNA fragments of interest, with each chromosome of interest represented by an individual line, such as in the graph in FIG. 2A.
   create an average profile based on all calculated chromosomes, as in the graph in FIG. 2B.
   If a given peak is not smooth, has multiple local minima, is noisy, or does not have a clear Gaussian shape, it is disregarded. For example, in FIG. 2B, we can clearly detect the locations of summits the seven first peaks.
   If the peak passes quality control (smooth, Gaussian distribution), determine its location. For example, the coordinates of the seven first peaks in FIG. 2B are as follows: 1) 187.2; 2) 377.4; 3) 571.6; 4) 759.8; 5) 958.0; 6) 1142.2; 7) 1336.3 (see FIG. 3A)
   make a scatter plot and perform linear regression to determine the slope of the line that gives the NRL (see FIG. 3B), in the example depicted in FIGS. 2 and 3 the NRL=191.5+/−0.5 (here we used precision of 0.1 bp).

As appropriate, repeat the above steps for each cfDNA sample (including reference samples from healthy people).

The method of calculating the histogram of nucleosome dyad-dyad distances is based on published software NucTools (https://generegulation.org/nuctools) and NRL_calc (https://github.com/chrisclarkson/NRLcalc).

### Calculation of NRL for Lower Sequencing Coverage.

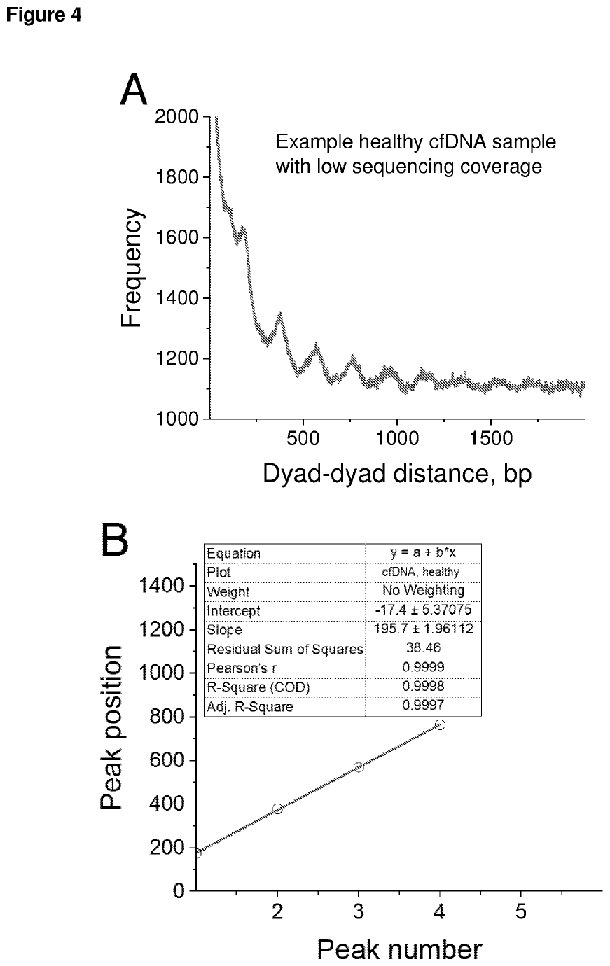

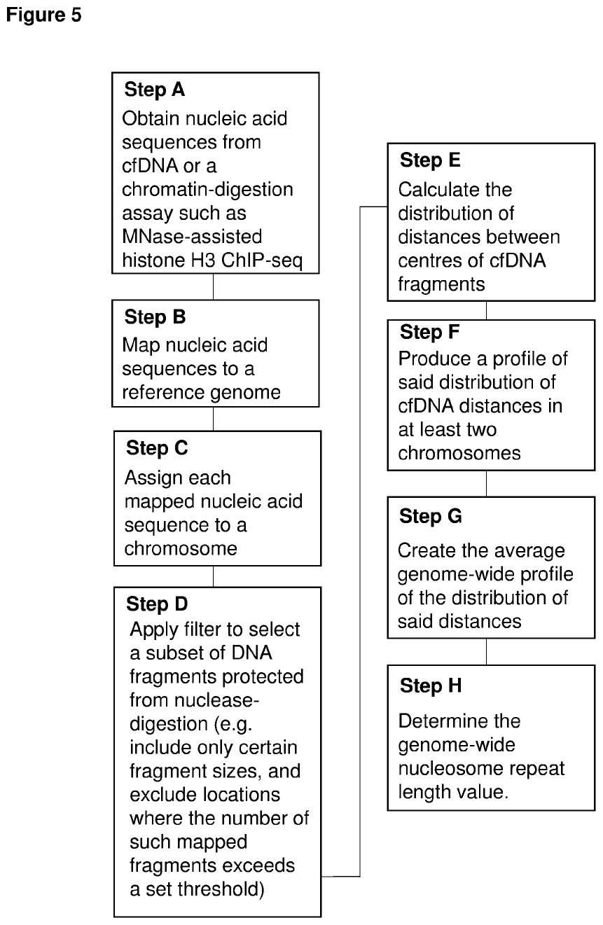

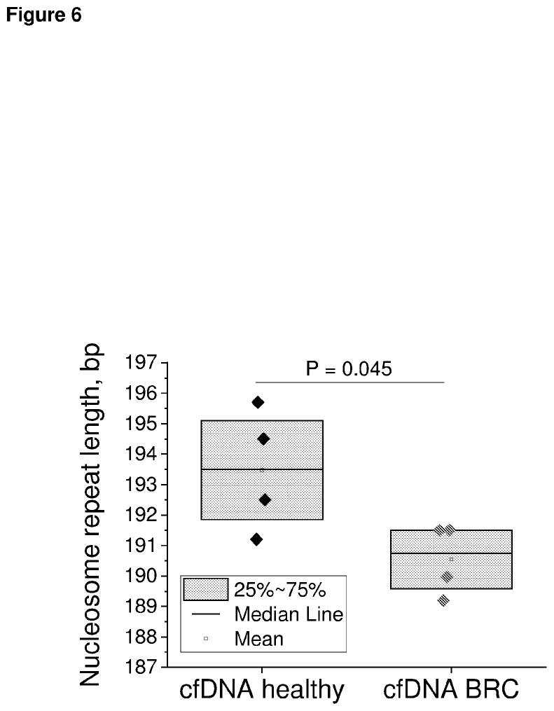

FIGS. 4A and B shows an example of genome-wide NRL definition for a reference healthy sample. Since the sample in this example has significantly lower sequencing coverage (shallow sequencing), the cfDNA distance distribution profile is noisier (FIG. 4A), and only locations of the summits of the first four peaks can be unambiguously determined without additional analysis such as smoothing and/or Fourier transformation (detailed below). In this case, the NRL determined with the linear regression method is defined as 195.7+/−2 bp.
The precision of defining the reference NRL can be improved by averaging over larger number of healthy samples (see FIG. 6), as well as applying additional mathematical approaches such as smoothing the cfDNA distance distribution profile and performing Fourier transformation to detect all major periodicities in the distribution.

### Example 2—Genome-Wide Nucleosome Repeat Length Analysis to Distinguish Breast Cancer Versus Normal Samples

Certain cancers are characterised by the decrease of nucleosome repeat length in tumour cells versus healthy cells of the same type, and these nucleosome repeat length differences can be detected in cfDNA.

The nucleosome repeat length for four healthy controls and four breast cancer patients was determined as detailed in Example 1. As depicted in FIG. 6, the cfDNA from breast cancer patients displayed a significant decrease in nucleosome repeat length (P=0.045, two-sample t-test).

Thus, nucleosomal DNA from tumour tissues enters body fluids and can be readily detected and analysed in cfDNA samples.

### Example 3—Genome-Wide Nucleosome Repeat Length Analysis to Distinguish Chronic Lymphocytic Leukaemia (CLL) Versus Normal Samples

The nucleosome repeat length for healthy subjects, and subjects with IGHV-mutated chronic lymphocytic leukaemia (M-CLL) and IGHV-unmutated chronic lymphocytic leukaemia (U-CLL), was determined as detailed in Example 1.

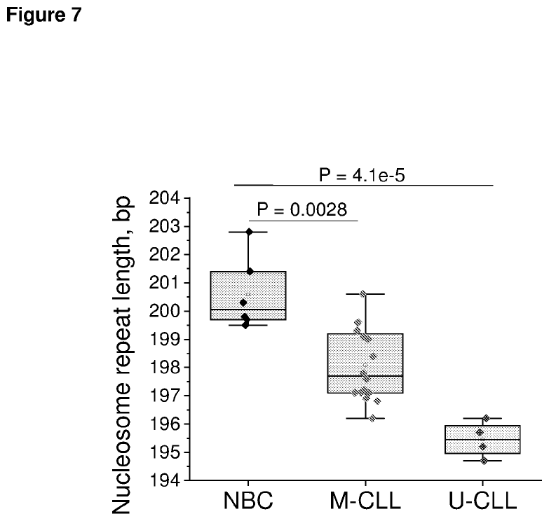

FIG. 7 demonstrates that nucleosome repeat length is significantly decreased in M-CLL compared to non-malignant B-cells from healthy people (NBCs) (P=0.0028). The reduction in nucleosome repeat length was even more significant in U-CLL compared to NBCs (P=4.1×10⁻⁵).

In particular the data depicted in FIG. 7 shows that genome-wide nucleosome repeat length decreases from ˜200 bp in NBCs to ˜198 bp and ˜195 bp in M-CLL and U-CLL, respectively.

### Example 4—Chromosome-Wide Nucleosome Repeat Length Analysis Allows Diagnostics of Breast Cancer

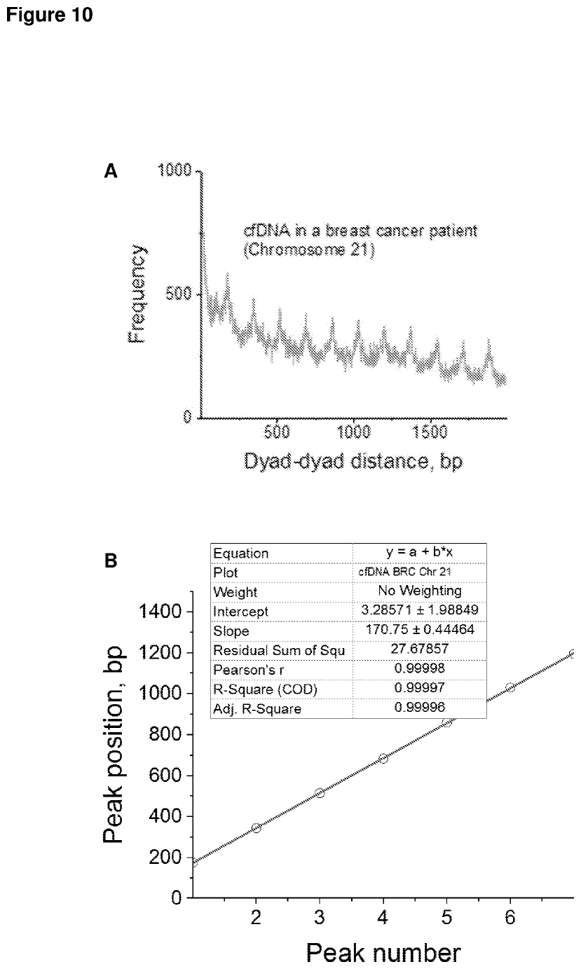

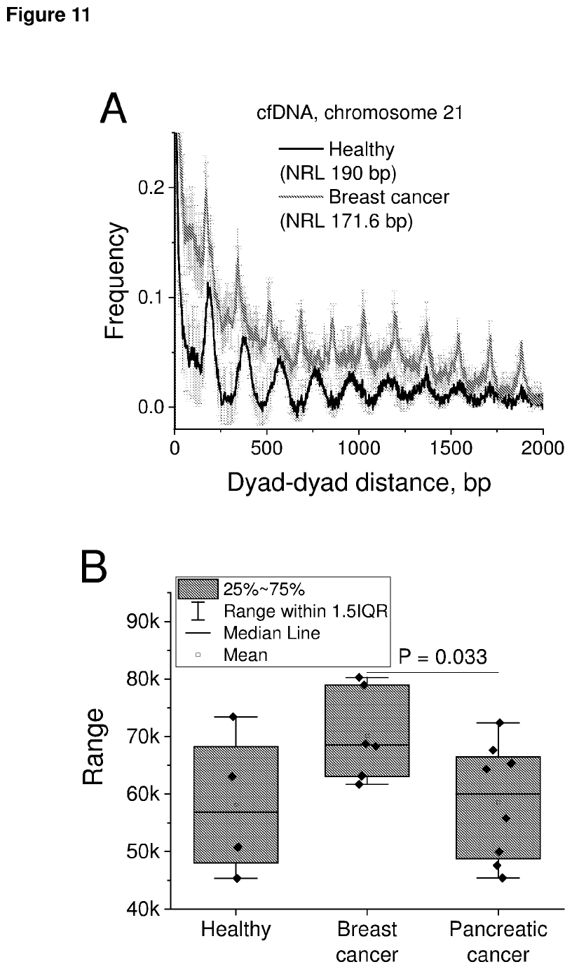

Calculations setup through to pre-processing was carried out as described in Example 1. Nucleosome repeat lengths for selected chromosomes (chromosome 21 in this example) were determined as follows:
1. Select a fraction of cfDNA fragments of interest. For example, only DNA fragments with sizes 120-180 bp were considered in the present Example.
2. Calculate for each sample the histogram of nucleosome dyad-dyad distances using NucTools as follows:
   split the BED file with all mapped paired-end reads into individual chromosomes and run the calculation in a loop, separately for each chromosome (autosomal chromosomes plus chromosomes X and Y)
   NucTools script nucleosome_repeat_length.pl is run with parameters --delta=2000 --MaxNr=10000000 --apply_filter
   Parameter --delta=2000 sets the maximum distance from start of the reference nucleosome (DNA fragment) to the last one in the calculations
   Parameter MaxNr=10000000 sets the maximum number of such reference nucleosomes considered per chromosome.
   The use of parameter “--apply_filter” means that nucleosome with piles more than 20 are removed from the analysis (genomic locations which have more than 20 DNA fragments centered at them).
3. Determine positions of the peak summits on the distribution of cfDNA distances and use these for linear regression to calculate NRL, as follows:
   create a graph with a selected chromosome represented by an individual line (as in FIG. 10A), or average profiles over the same chromosomes for several samples (as in the graph in FIG. 11A).
   If a given peak is not smooth, has multiple local minima, is noisy, or does not have a clear Gaussian shape, it is disregarded.
   If the peak passes quality control (smooth, Gaussian distribution), determine the location of its summit.
   make a scatter plot using the locations of the peak summits and perform linear regression to determine the slope of the line that gives the NRL, in the example depicted in FIG. 10B.

In FIG. 11A, the distributions of cfDNA distances for chromosome 21 from individual samples were averaged over 6 breast cancer samples (SRA accession numbers SRR2130045, SRR2130043, SRR2130033, SRR2130032, SRR2130011, SRR2130004), and separately 4 healthy samples (SRA accession numbers SRR2130050, SRR2130051, SRR2130052, 21229993). the nucleosome repeat length for chromosome 21 was then calculated separately for the healthy and breast cancer profiles. Breast cancer samples displayed a reduced nucleosome repeat length compared to healthy samples—with values of 171.6 bp and 190 bp, respectively. This constitutes a difference of about 18 bp, which is a very large and easily detectable difference that allows to classify correctly all these 10 samples as being healthy or breast cancer.

The difference in chromosome 21 nucleosome repeat length between breast cancer patients and healthy people is further exemplified by FIG. 11A, which shows the dyad-dyad distances from cfDNA samples (N=10 samples). In particular all the breast cancer samples displayed similar short nucleosome repeat length values (around 171-172 bp). Thus, the diagnostics of patients based on this feature is 100% effective for this dataset.

### Example 5—The Relative Number of cfDNA Mapped to Alpha-Satellite Repeats Allows Distinguishing Pancreatic and Breast Cancer

The short NRL=171-172 bp observed for chromosome 21 in breast cancer samples in Example 4 above can be explained by the increased number of cfDNA fragments that map to alpha-satellite repeats (sometimes referred to as the centromeric repeats). The periodicity of alpha satellite repeats coincides with the NRL value determined for the cancer samples in FIGS. 10A and 11A. This suggests that in some situations the number of cfDNA fragments mapped to alpha-satellite repeats in different genomic loci, as well as other types of repeats, can be used as a diagnostic marker per se. FIG. 11B demonstrates this for the normalised amounts of cfDNA fragments mapped to alpha-satellite repeats (chr1:121,480,151-121,485,429) per 10,000,000 reads for four healthy and six breast cancer cfDNA samples from FIG. 11A, as well as eight cfDNA samples from patients with pancreatic cancer (accession numbers SRR2130020, SRR2130023, SRR2130024, SRR2130044, SRR2130046, SRR2130047, SRR2130048, SRR2130049). For each sample, the number of cfDNA fragments mapped to alpha-satellite repeats per 10,000,000 of total uniquely mapped cfDNA fragments was determined using cfDNA reads mapped to the human hg19 genome.

The difference between the number of cfDNA fragments mapped to alpha-satellite repeats in pancreatic cancer and breast cancer samples is statistically significant (two sample t-test, not assuming equal variance (Welch correction), P=0.0328; two-sample t-test assuming equal variance, P=0.03886).

The calculation described in this example was performed using command analyzeRepeats.pl of software HOMER (Heinz S, Benner C, Spann N, Bertolino E et al. (2010) Simple Combinations of Lineage-Determining Transcription Factors Prime cis-Regulatory Elements Required for Macrophage and B Cell Identities. Mol Cell 38, 576-589).

This example shows a method to classify samples for the purpose of diagnostics, monitoring or stratification based on a number of cfDNA repeats mapped to alpha-satellite repeats. This method is not limited to alpha-satellite repeats as different types of repeats (e.g. ALU, L1, etc) can be overrepresented in cfDNA in cancer. Furthermore, this method is not limited to breast cancer and pancreatic cancer, since different repeats may be overrepresented in cfDNA in different diseases.

The output of the HOMER command analyzeRepeats.pl used above contains normalised values for cfDNA reads mapped to 1397 types of different genomic repeats (of which only one type of subtype of alpha-satellite repeats was used to generate FIG. 11B). This data can be used for a method of sample classification based on machine learning, where each sample is characterised by the input vectors of 1397 values corresponding to 1397 types of different genomic repeats considered in the HOMER software used above. Another variation of this method includes creating a panel of genomic repeats and calculating normalised numbers of cfDNA fragments mapped to each of these repeats for a given sample. Such vector of values corresponding to normalised numbers of cfDNA fragments.

### Example 6—Chromosome-Wide Nucleosome Repeat Length Analysis Allows Diagnostics of Chronic Lymphocytic Leukaemia (CLL)

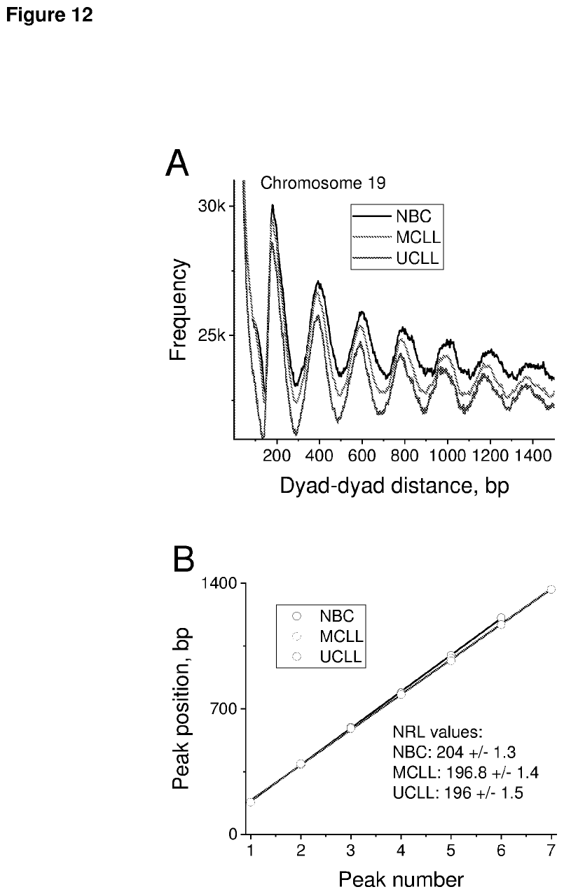

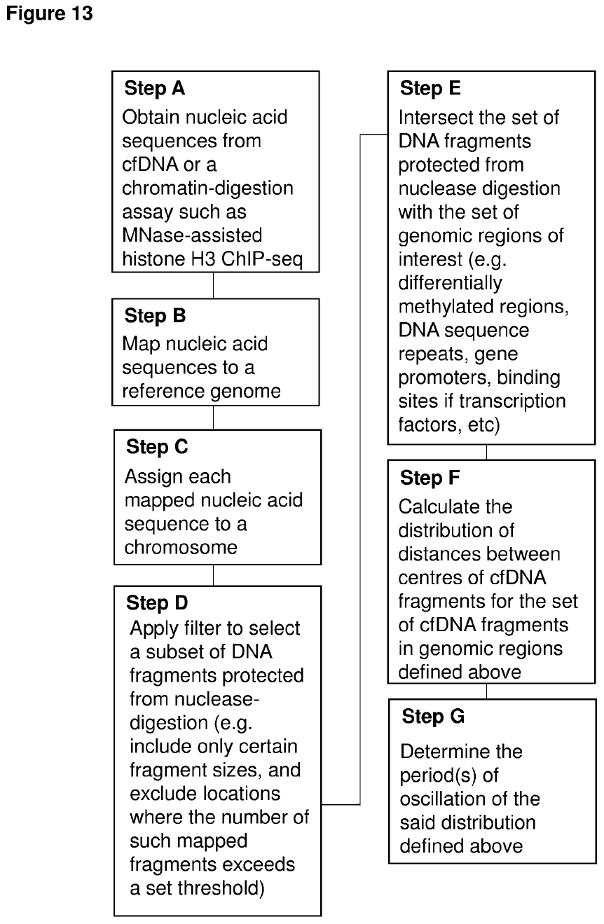

The nucleosome repeat length of chromosome 19 was determined in healthy subjects, and subjects with IGHV-mutated chronic lymphocytic leukaemia (M-CLL) and IGHV-unmutated chronic lymphocytic leukaemia (U-CLL), as detailed in Example 4. FIG. 12 demonstrates that chromosome-wide nucleosome repeat length for chromosome 19 decreases from ˜204 bp in non-malignant B-cells from healthy people (NBCs) to ˜197 bp and ˜196 bp in M-CLL and U-CLL, respectively. Therefore, individual chromosome 19 appears a characteristic marker for CLL because it has a larger NRL in healthy B-cells.

### Example 7—Nucleosome Repeat Length Determined Around Differentially Methylated Regions (DMR) Allows Diagnostics of Chronic Lymphocytic Leukaemia (CLL)

In this example, the nucleosome repeat length was determined in healthy subjects, and subjects with IGHV-mutated chronic lymphocytic leukaemia (M-CLL) and IGHV-unmutated chronic lymphocytic leukaemia (U-CLL), as detailed in Examples 4 and 6, but now the NRL calculation was limited to the differentially methylated regions (DMR), that are characterised by differential DNA methylation in CLL.
The list of DMRs was downloaded from [Mallm J.-P., Iskar M., Ishaque N., Klett L. C., Kugler S. J., Muino J. M., Teif V. B., Poos A. M., Großmann S., Erdel F., Tavernari D., Koser S. D., Schumacher S., Brors B., König R., Remondini D., Vingron M., Stilgenbauer S., Lichter P., Zapatka M., Mertens D., Rippe K. (2019) Linking aberrant chromatin features in chronic lymphocytic leukemia to transcription factor networks. Mol Syst Biol 15, e8339].
This list of DMRs was extended by 10,000 bp on both sides of the original DMR intervals.

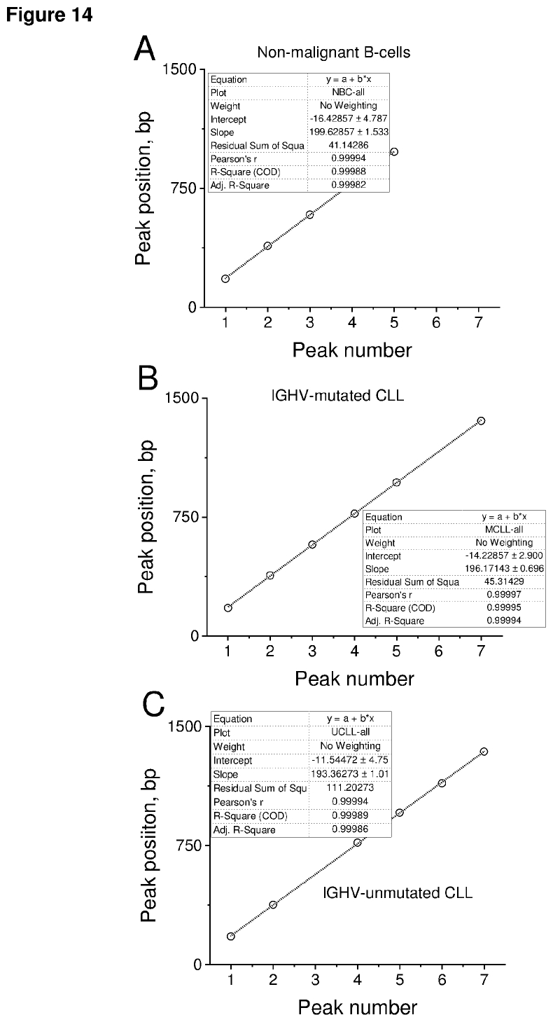

For each patient, the mapped nucleosome locations determined with MNase-assisted histone H3 ChIP-seq from Mallm et al were intersected using BedTools with the 10,000 bp-extended DMR regions. The intersection was considered valid if at least one base pair overlapped. The nucleosome locations in these regions were then used for the analysis to determine NRL following the same remaining workflow as in Examples 3 and 7, but limiting the analysis to the extended DMR regions.
FIG. 14 demonstrates that near DMRs, the nucleosome repeat length decreases from ˜200 bp in non-malignant B-cells from healthy people (NBCs) to ˜196 bp and ˜193 bp in M-CLL and U-CLL, respectively. Therefore, this set of genomic regions can be used as a characteristic marker for CLL because it has a smaller NRL in U-CLL.

Importantly, Examples 3, 6 and 7 consistently observed that U-CLL (more aggressive CLL subtype) has smaller NRL than M-CLL (less aggressive CLL subtype). Therefore, this analysis can be used not only to distinguish healthy from cancer, but also to stratify subclasses of cancer by their aggressiveness/stage/response to therapy.

### Example 8—Nucleosome Repeat Length Determined in Genomic Regions Around L1 Repeats Allows Diagnostics of Liver Cancer and Colorectal Cancer

In this example, NRL was calculated inside genomic regions around L1 repeats, separately for a reference set composed of four healthy cfDNA samples, for a cfDNA sample from a patient with liver cancer and for a cfDNA samples with colorectal cancer.

The following cfDNA samples were used in this analysis: four healthy cfDNA samples (SRA accession numbers SRR2130050, SRR2130051, SRR2130052, 21229993), cfDNA sample from a patient with liver cancer (SRA accession number SRR2130016) and cfDNA from a patient with colorectal cancer (SRA accession number SRR2130035).
Genomic locations of L1 repeats in the human genome hg19 were determined with the UCSC genome masker
The BED file with the repeat masker was downloaded from https://genome.ucsc.edu
Regions containing L1 repeats were extracted using Linux grep command.
Regions with L1 repeats were extended by 1,000 bp using BEDtools command Slop.
cfDNA fragments uniquely mapped to hg19 genome with Bowtie allowing up to two mismatches were intersected with the extended regions containing L1 repeats. The intersection was considered valid if at least one bp was overlapping between two regions.
cfDNA fragments intersecting with extended L1 repeats as defined above were used for the calculation of the distribution of distances between cfDNA fragments.
Such distributions were calculated separately for healthy, liver cancer and colorectal cancer samples.
In the case of healthy samples, the profile was averaged over four samples SRR2130050, SRR2130051, SRR2130052; 21229993.
In the case of liver cancer and colorectal cancer, profiles were calculated for each individual sample.

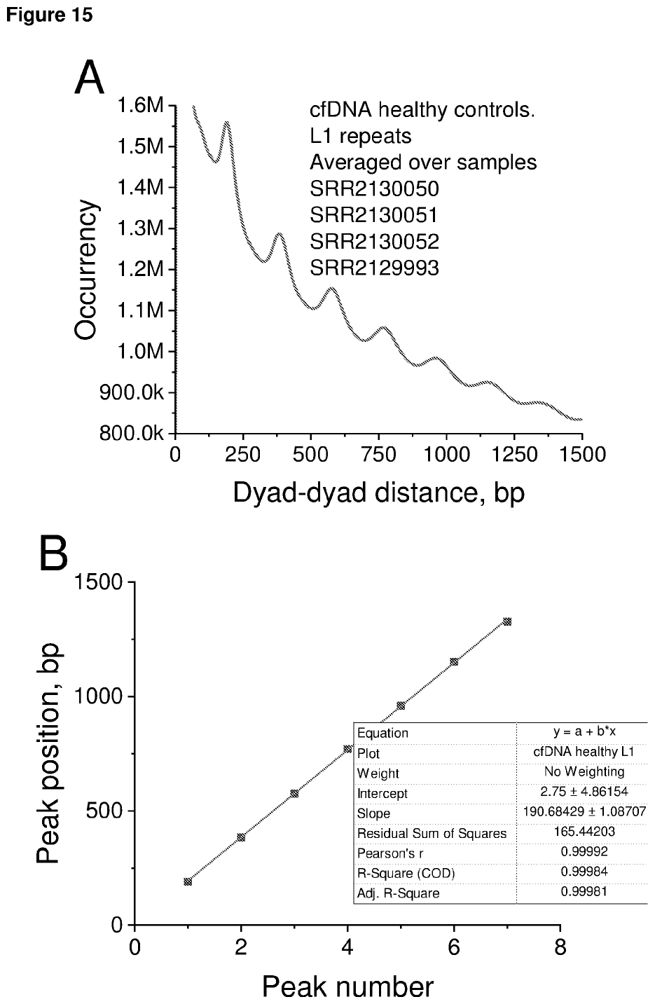

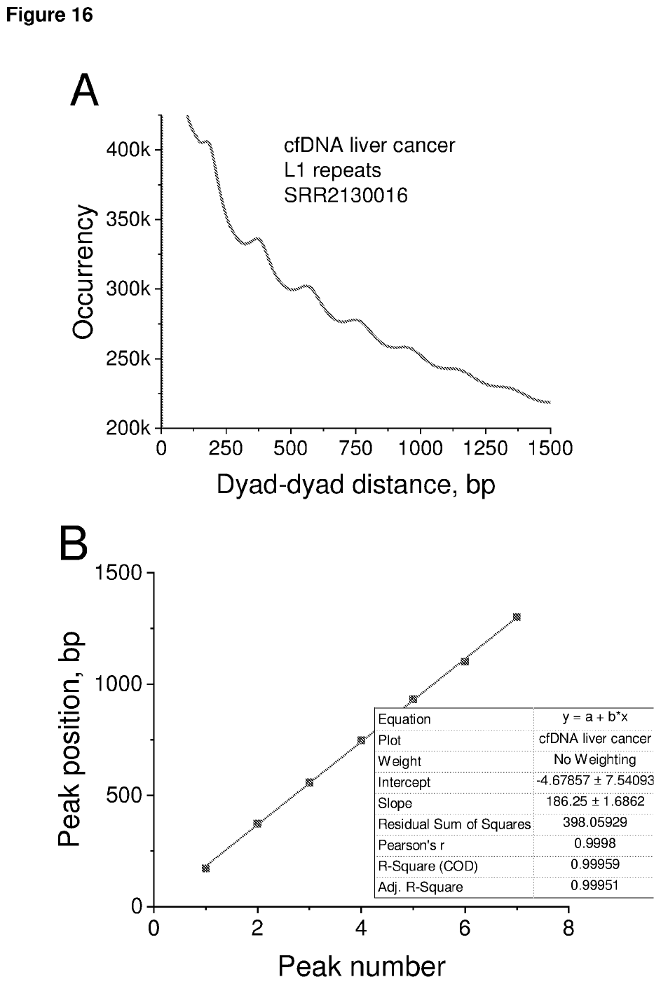

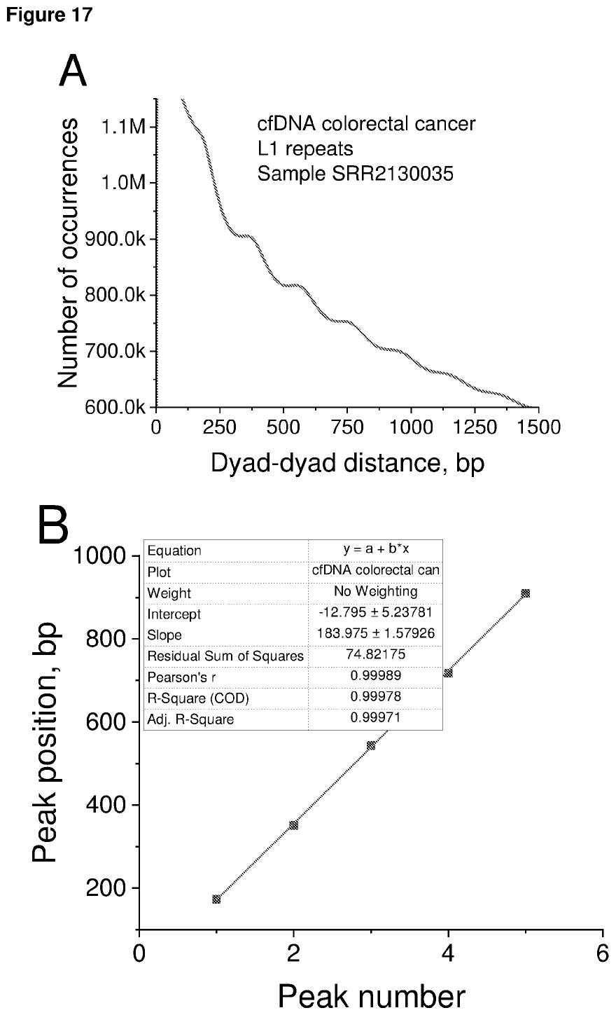

The results of these calculations are shown on FIG. 15 (healthy), FIG. 16 (liver cancer) and FIG. 17 (colorectal cancer). In this example, the NRL inside genomic regions around L1 repeats changed from about 190.7 bp in healthy controls to 186.3 bp in liver cancer and 184 bp in colorectal cancer.

### Example 9—Fourier Transformation Allows Distinguishing Cancer Versus Normal Samples Based on Different Frequencies in the Distribution of Distances Between DNA Fragments Protected From Nuclease Digestion

Fourier transformation (Fourier transform) is a term applied here to the group of mathematical methods including Discrete Fourier Transformation (DFF) and Fast Fourier Transformation (FFT), as explained at the web site of the Origin software used for the calculations in this example (https://www.originlab.com/doc/Origin-Help/FFT).

Fourier transformation provides an alternative method of NRL calculation, complementary to the method of linear regression used in the examples above. The NRL values obtained with the methods of Fourier transformation may be different from the NRL values obtained with the linear regression method. Therefore, the comparison across different biological samples should be carried out systematically using either the linear regression or the Fourier transformation method. The example below shows that in certain situations the Fourier transformation method allows effective discrimination between different healthy and diseased samples.

### Example 9A—Application of the Fourier Transformation Method to Calculate NRL Near Binding Sites of a Chromatin Protein CTCF in the Breast Cancer Cell Line MCF-7

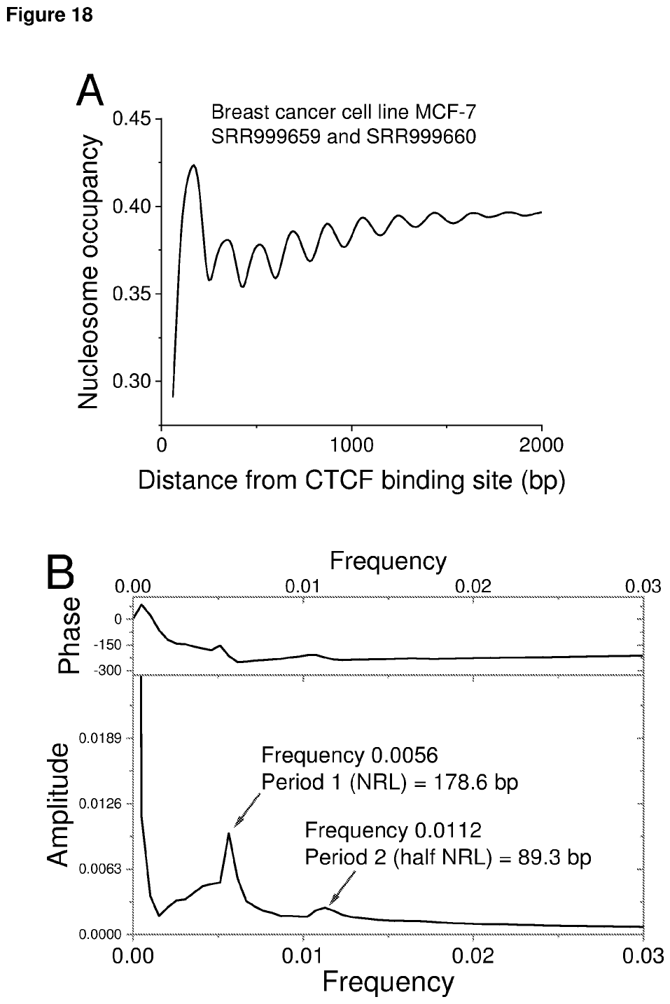

A. Coordinates of CTCF sites in the human genome assembly hg19 based on the ChIP-seq experiments performed by the ENCODE consortium were obtained from the GEO database (GSM822305) in the form of the file “GSM822305_hg19_wgEncodeOpenChromChipMcf7CtcfPk.narrowPeak.gz”.
B. MNase-seq sequencing data in MCF-7 cells was obtained from GEO entry GSE51097, specifically from two SRA entries: SRR999659 and SRR999660. MNase-seq reads from these two entries were mapped separately, then merged and the merged dataset was used to generate HOMER tag directories.
C. Nucleosome occupancy profiles were calculated based on MNase-seq in MCF-7 cells detailed above, around CTCF binding sites in MCF-7 detailed above, using HOMER script annotatePeaks.pl within the interval [0, 2000] from the center of CTCF site, with a 10-bp step, using human genome hg19. The resulting profile is shown in FIG. 18A.
D. Fast Fourier Transformation (FFT) of the profile shown in FIG. 18A was performed with software Origin Pro (Origin Labs) (https:/www.originlab.com/) using the following parameters:
   Window: rectangular;
   Window correction: amplitude;
   Normalise Re, Im, Mag: no;
   Shift: yes;
   Unwrap phase: yes;
   Factor: −1;
   Spectrum type: auto;
   Normalise power to: MSA—mean square amplitude;
   Preview: amplitude/phase.
E. The detailed description of these parameters is provided at the following page: https:/www.originlab.com/doc/en/Origin-Help/FFT1-Dialog
F. FIG. 18B shows the results of the Fast Fourier Transformation analysis performed for this dataset. The upper panel in FIG. 18B shows the FFT phase as a function of frequency. The lower panel shows the FFT amplitude as a function of frequency.
G. The amplitude vs frequency graph from FIG. 18B is used to determine the prevalent frequencies in the nucleosome occupancy profile around CTCF binding sites a given sample. At least two peaks can be clearly observed. The first peak at frequency 0.0056 determines the period equal to 1/0.0056=178.6. This corresponds to the NRL=178 bp, which is consistent with previous estimates of NRL near CTCF in mouse cells performed using the linear regression method [Clarkson et al. (2019), Nucleic Acids Res 47, 11181-11196]. The second peak with frequency 0.00112 determines the period equal to 1/0.00112=89.3, which is equal to the half of the NRL value defined above.

### Example 9B—Application of the Fourier Transformation Method to Calculate the Periods of the Patterns of Distributions of Distances Between cfDNA Fragments to Distinguish Between Healthy and Breast Cancer cfDNA Samples

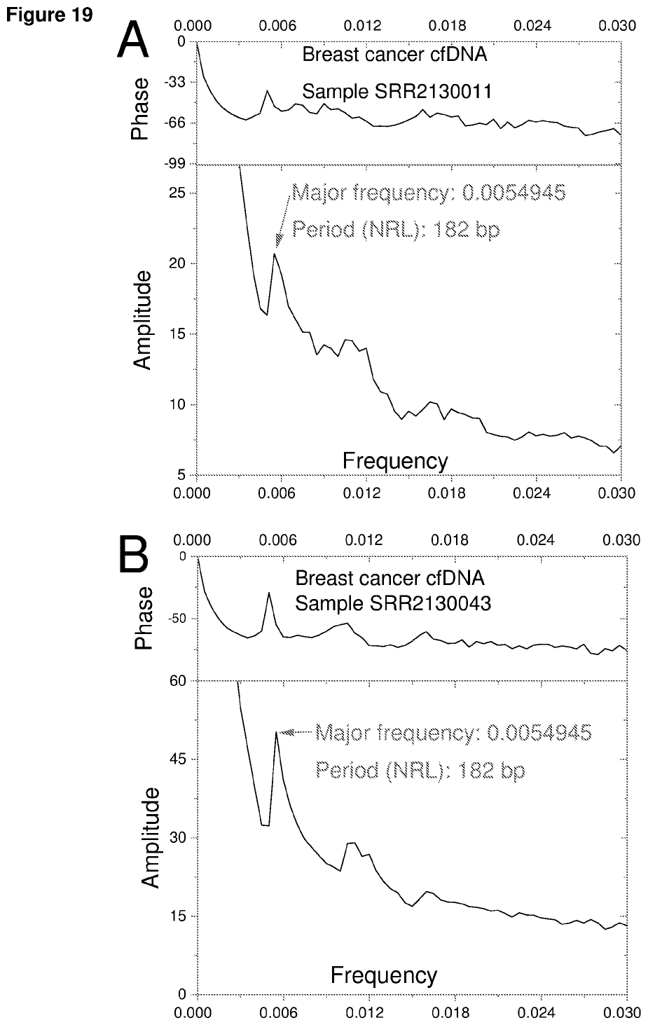

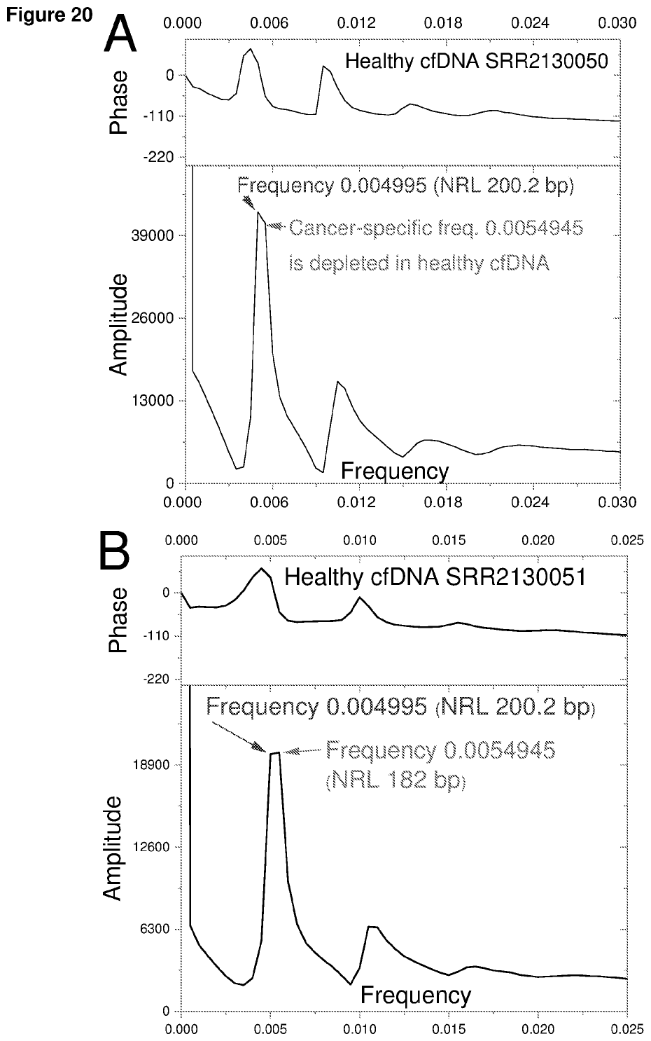

A. Fast Fourier Transformation (FFT) method described in steps (D)-(G) of Example 9A was applied to the genome-wide distributions of distances between cfDNA samples from four cfDNA samples from two breast cancer patients and two healthy controls.
B. FIG. 19 shows the application of this method to two breast cancer cfDNA samples (FIG. 19A, SRA accession number SRR2130011; FIG. 19B, SRA accession number SRR2130043).
C. In both breast cancer cfDNA samples shown in FIGS. 19A and 19B, the first amplitude peak is located at frequency 0.0054945. The NRL value estimated based on this frequency is equal to 182 bp for both these samples (1/0.0054945=182).
D. Note that this NRL value is smaller than the genome-wide NRL values determined with the linear regression method for healthy cfDNA samples in previous examples. However, the Fourier transform method and the linear regression method may give different absolute values of NRLs, therefore, a comparison needs to be carried out for NRLs determined with Fourier transform method for healthy cfDNA samples (see next step).
E. FIG. 20 shows the application of this method to two healthy cfDNA samples (FIG. 20A, SRA accession number SRR2130050; FIG. 20B, SRA accession number SRR2130051).
F. In both healthy cfDNA samples shown in FIGS. 20A and 20B, the first peak has “rectangular” shape corresponding to two frequencies.
G. In FIG. 20A, healthy cfDNA sample SRR2130050 is characterised by the maximum amplitude of the FFT spectrum at frequency 0.004995. This corresponds to the NRL value 200.2 bp (1/0.004995=0.004995). Note that the value of NRL around 200 bp is consistent with NRL in healthy B-cells (see Example 3 above) which typically represent the source of most cfDNA fragments in healthy people. In addition to this “healthy” NRL marker, this sample also has a second FFT frequency 0.0054945. The NRL value estimated based on this frequency is equal to 182 bp (1/0.0054945=182), which is a marker of breast cancer cfDNA based on FIG. 19. But the latter “cancer-associated” frequency is less prevalent in this sample in comparison with the more prevalent FFT frequency determining NRL=200.2 bp.
H. FIG. 20B shows similar analysis as in the previous step, conducted for another healthy sample (SRA accession number SRR2130051). In this case the amplitude of the “cancer-specific” NRL=182 bp is comparable to the amplitude of the “healthy” NRL=200.2 bp.
I. It is clear that in both examples in FIGS. 20A and 20B, there is a significant presence of the “healthy” FFT frequency determining NRL at 200.2 bp, which is complemented by the existence of the “cancer-specific” FFT frequency determining NRL=182 bp. This is different from the cancer cfDNA samples in FIGS. 19A and 19B, which have only one “cancer-specific” FFT frequency determining NRL=182 bp. Thus, the FFT analysis allows distinguishing healthy and cancer samples.

It is worth noting that the FFT analysis described above allows determining a significantly large number of FFT frequencies and associated NRL values, which can be used to construct a unique marker of a given sample.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of determining a genome-wide distribution of genomic distances between DNA fragments protected from nuclease digestion, the method comprising:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a first subset of the cfDNA fragments, each cfDNA fragment aligning to a first chromosome;
(e) selecting a further subset of cfDNA fragments, which align to a second chromosome;
(f) calculating the distribution of frequencies of distances between cfDNA fragments of:
(fi) the first subset within a pre-determined genomic distance range from each other to form a distribution of frequencies of cfDNA distances from the first chromosome, and
(fii) the second subset within a pre-determined genomic distance range from each other to form a distribution of frequencies of cfDNA distances from the second chromosome;
(g) averaging the distribution of frequencies of cfDNA distances across the first and second chromosomes to create a distribution of frequencies of cfDNA distances across multiple chromosomes; and
(h) analysing the distribution of frequencies of cfDNA distances to detect periodic patterns and calculating at least one periodicity parameter.

2. The method of claim 1, wherein:
(I) the method further comprises using said distribution as a marker of a disease or healthy condition;
(II) the method further comprises (i) using the distribution of frequencies of cfDNA distances or its parts or periodicity parameters derived from it as a marker of a disease or healthy state to perform cfDNA sample classification;
(III) the at least one periodicity parameter is selected from a period(s) of oscillation of the distribution of frequencies of cfDNA distances, and/or relative numbers of cfDNA fragments mapped to different types of genomic DNA sequence repeats;
(IV) the DNA fragments are protected from nuclease digestion by a nucleosome, other DNA-bound nucleoprotein complex or a sequence-dependent DNA structure;
(V) the method further comprises (j) selecting one or more further subsets of cfDNA fragments, each further subset of cfDNA fragments aligning to a corresponding further chromosome, wherein the distribution of frequencies of cfDNA distances across multiple chromosomes is created from all the selected subsets of cfDNA; or
(VI) the genome-wide period of oscillation of the distribution of frequencies of cfDNA distances is a nucleosome repeat length (NRL) value.

3.-7. (canceled)

8. The method of claim 1, wherein:
(I) step (h) comprises performing Fourier transform, discrete Fourier transform, fast Fourier transform or equivalent methods that decompose the distribution of frequencies of cfDNA distances to determine one or several periods of oscillation of distributions of frequencies of cfDNA distances, the method comprising:
(a) calculating the distribution of distances between DNA fragments protected from nuclease digestion, or
(b) calculating the distribution of the probabilities that a given genomic location represents the center of a nucleosomes or is covered by a nucleosome (so called aggregate nucleosome profiles), around genomic features such as transcription start sites, transcription factor binding sites, transcription termination sites, nucleosome depleted regions or stably positioned nucleosomes;
(c) calculating the Fourier transform (FT), discrete Fourier transform (DFT) or fast Fourier transform (FFT) of one of the said distributions;
(d) determining the prevalent frequencies of the said Fourier transform or equivalent transformations based on the peaks of the corresponding distribution of the transformation amplitudes as a function of the corresponding transformation frequencies;
(e) determining the values of the nucleosome repeat length (NRL) and other periods of oscillation of the original distributions defined in steps (a-b) as the inverse value of the said frequencies defined in step (d); or
(II) step (h) comprises performing linear regression on values corresponding to the locations of the summits of the peaks of the frequency distributions of cfDNA distances, to calculate the genome-wide nucleosome repeat length value (NRL).

9. (canceled)

10. A method of determining a chromosome-wide distribution of genomic distances between DNA fragments protected from nuclease digestion, the method comprising:
(a) providing a plurality of nucleic acid sequences, wherein the nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence to a genomic location, wherein each mapped nucleic acid sequence is a cfDNA fragment;
(d) selecting a subset of cfDNA fragments, each of which aligns to a first chromosome residing in a genomic region of interest;
(e) calculating the distribution of frequencies of distances between cfDNA fragments of the subset of cfDNA fragments within a pre-determined distance range from each other; and
(f) analysing the said distribution of frequencies of cfDNA distances to detect periodic patterns and calculating at least one periodicity parameter.

11. The method of claim 10, wherein:
(I) the method further comprises using said distribution as a marker of a disease or healthy condition;
(II) the method further comprises (g) using the distribution of frequencies of cfDNA distances or its parts or periodicity parameters derived from it as a marker of a disease or healthy state to perform cfDNA sample classification;
(III) the at least one periodicity parameter is selected from a period(s) of oscillation of the distribution of frequencies of cfDNA fragments, and/or the relative numbers of cfDNA fragments mapped to different types of DNA sequence repeats;
(IV) step (f) comprises performing linear regression on the coordinates of the summits of the peaks of the frequency distributions of cfDNA distances to calculate the NRL value; or
(V) the chromosome-wide period of oscillation of the distributions of frequencies of cfDNA distances is a nucleosome repeat length (NRL) value.

12.-14. (canceled)

15. A method of determining a distribution of genomic distances between DNA fragments protected from nuclease digestion, the method comprising:
(a) providing a plurality of nucleic acid sequences, wherein the plurality of nucleic acid sequences are obtained from cell free DNA (cfDNA) present in a sample obtained from a subject or from a database;
(b) aligning each of the plurality of nucleic acid sequences to a reference genome or portion thereof to obtain a plurality of mapped nucleic acid sequences;
(c) assigning each mapped nucleic acid sequence, wherein each mapped nucleic acid sequence is a cfDNA fragment, to a genomic location;
(d) selecting a subset of cfDNA fragments, each of which aligns to a region of interest in a first chromosome,
(e) calculating the distribution of frequencies of distances between cfDNA fragments of the subset of cfDNA fragments within a pre-determined distance range from each other within the genomic regions of interest, to form a distribution of frequencies of cfDNA distances; and
(f) analysing the said distribution of frequencies of cfDNA distances to detect periodic patterns and calculating at least one periodicity parameter.

16. The method of claim 15, wherein;
(I) the method further comprises using said distribution or its parts of periodicity parameters derived from it as a marker of a disease or healthy condition;
(II) the method further comprises (g) using the distribution of frequencies of cfDNA distances or its parts or periodicity parameters derived from it as a marker of a disease or healthy state to perform cfDNA sample classification;
(III) the at least one periodicity parameters is selected from a period(s) of oscillation of the distribution of frequencies of cfDNA distances and/or the relative numbers of cfDNA fragments mapped to different types of DNA sequence repeats;
(IV) the region of interest is selected from a region or a plurality of regions such as DNA sequence repeats, a set of binding sites of a transcription factor, a gene promoter and a region of differential DNA methylation;
(V) the period of oscillation of the distribution of frequencies of cfDNA distances within the genomic regions of interest is a nucleosome repeat length (NRL) value; or
(VI) step (d) comprises selecting of the region of interest based on the locations of gene bodies, enhancers, insulators, other regulatory genomic elements, binding sites of transcription factors, centromeric regions, heterochromatin regions, telomeric regions, DNA sequence repeats such as ALU, LINE, SINE, alpha-satellite repeats, microsatellite repeats, other types of DNA sequence repats, different types of chromatin domains such as topologically associating domains (TADs), lamina associated domains (LADs) or other types of domains, and/or genomic regions with enriched binding of different chromatin proteins and/or RNAs and/or regions with low/high/condition-sensitive DNA methylation or another epigenetic modification.

17-21. (canceled)

22. The method according to claim 1, wherein:
(I) the distance between cfDNA fragments is calculated based on:
(i) the distribution between genomic coordinates of the centers of cfDNA fragments; and/or
(ii) the distribution between genomic coordinates of the edges of cfDNA fragments;
(II) the biomolecular complexes protecting DNA from nuclease digestion are nucleosomes; or;
(III) the reference genome is a human genome, optionally the reference genome is GRCh37/hg19, T2T CHM13, GRCh38/hg38 or another human genome, or any animal genome, or any other genome.

23.-24. (canceled)

25. The method according to claim 1, comprising selecting the first and optionally further subsets of cfDNA fragments based on one or more of the following:
(i) a predetermined length range of cfDNA fragments;
(ii) inclusion of one or more locations where the number of such mapped fragments exceeds a set threshold, which depends on the sequencing coverage of a given sample and
(iii) exclusion of locations where the number of such mapped fragments exceeds a set threshold, which depends on the sequencing coverage of a given sample.

26. The method of claim 25 wherein the predetermined length range of cfDNA fragments is between 10-10000 base pairs (bp), and is optionally 100-200 bp or 10-300 bp.

27.-30. (canceled)

31. A method of determining a subject's disease state using genome-wide nucleosome spacing, the method comprising:
(a) determining genome-wide sizes of multiple-nucleosome fractions, or distances between nucleosomes, or NRL, or other nucleosome periodicity parameters derived from these for a subject in at least one timepoint, according to the method of claim 1; and
(b) comparing the determined value to at least one set of reference nucleosome repeat length values;
wherein a time-dependent change of the determined sizes of multiple-nucleosome fractions, or distances between nucleosomes, or NRL, or other nucleosome periodicity parameters derived from these or a match to any reference values of these parameters indicates a presence or absence of a disease or a specific state of healthy functioning.

32. The method of claim 31, wherein;
(I) the NRL is 199-204 bp for non-malignant B-cells and is between 193-198 bp for B-cells in chronic lymphocytic leukemia (CLL), optionally wherein CLL subtype unmutated IGHV gene in general characterized by smaller NRL value than CLL subtype with mutated IGHV gene;
(II) the NRL of cfDNA in healthy people is approximately 190 bp and wherein the NRL of cfDNA obtained from a patient suffering from cancer is 169-173 bp in chromosome 21 and other chromosomes and genomic loci enriched with alpha-satellite repeats; or
(III) step (b) comprises comparing
(i) NRL determined using the same experimental method for the same subject at different time point(s);
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; and/or
(iv) NRL determined using the same experimental method for an age- and gender-matched cohort of healthy people.

33.-34. (canceled)

35. A method of determining a subject's disease state using chromosome-wide nucleosome spacing, the method comprising:
(a) determining a chromosome-wide NRL value for a subject in at least one timepoint on the method of claim 8; and
(b) comparing the determined value to at least one set of reference NRL values, wherein the time-dependent change of the determined NRL value or a match to any specific reference NRL values may indicate a presence or absence of a disease or a specific state of healthy functioning.

36. The method of claim 35, wherein step (b) comprises comparing one or more of the following:
(i) NRL determined using the same experimental method for the same subject at different time point(s);
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; and/or
(iv) NRL determined using the same experimental method for an age- and gender-matched cohort of healthy people.

37. The method of claim 36, wherein:
a) NRL is approximately 204 bp for chromosome 19 in non-malignant B-cells and is around 196 bp for chronic lymphocytic leukemia; and/or
b) in cfDNA of healthy people, NRL in chromosome 21 and other chromosomes enriched with alpha satellite repeats is around 190 bp and around 169-172 bp in a subject suffering from breast cancer in some genomic loci.

38. A method of determining a subject's disease state using nucleosome spacing in genomic regions of interest, the method comprising:
(a) determining the NRL value in a region of interest in at least one timepoint based on the method of claim 15; and
(b) comparing the determined NRL value to at least one set of reference NRL values, wherein the time-dependent change of the determined NRL value or a match to any specific reference NRL values may indicate a presence or absence of a disease or a specific state of healthy functioning.

39. The method of claim 38, wherein step (b) comprises comparing one or more of the following:
(i) NRL determined using the same experimental method for the same subject at different time point(s);
(ii) NRL determined using the same experimental method for the same subject at different age to monitor the health status of a subject;
(iii) NRL determined using the same experimental method for an age- and gender-matched cohort of patients with the same disease type as the one that is being monitored in the subject, to classify disease stage/progression/aggressiveness; or
(iv) NRL determined using the same experimental method for an age- and gender-matched cohort of healthy people.

40. The method of claim 39, wherein:
(a) NRL is around 200 bp for CLL-specific differentially methylated regions (DMR) in non-malignant B-cells and around 193 bp for aggressive types of chronic lymphocytic leukemia; and/or
(b) in cfDNA of healthy people, NRL in regions enclosing L1 DNA sequence repeats is around 191 bp and in patients with breast cancer and colorectal cancer NRL is around 188bp and in patients with liver cancer, optionally NRL in regions enclosing L1 DNA sequence repeats decreases to around 186 bp.

41. The method of claim 1 for the use in determining a subject's disease state using the calculation of the relative numbers of cfDNA fragments mapping to different types of DNA sequence repeats, the method comprising:
(a) providing a plurality of nucleic acid sequences, wherein the plurality of nucleic acid sequences are obtained from cell free DNA (cfDNA) present in a sample from a subject or from a database;
(b) aligning each of the plurality of nucleic acid sequences to a reference genome or portion thereof to obtain a plurality of mapped nucleic acid sequences;
(c) determining the number of cfDNA fragments aligning to at least one type of DNA sequence repeats;
(d) determining a relative frequency of the representation of different repeat subtypes/families in a given cfDNA sample by performing normalization of the number of cfDNA fragments aligning to each family of DNA sequence repeats per 10,000,000 mapped reads, or use another type of normalization that takes into account the sequencing coverage of a sample;
(e) comparing the frequency distribution of DNA sequence repeat subtypes of a sample with such distributions in other samples or in a reference database to perform sample classification using:
(i) a predefined linear model, or
(ii) a machine learning model based on the vector composed of the relative number of different families of DNA sequence repeats represented in a given sample.

42. The method of claim 1 for the use in determining a subject's disease state using machine learning techniques for the analysis of nucleosome spacing in genomic regions of interest, the method comprising:
(a) determining the distributions of frequencies of cfDNA distances based on claim 1;
(b) creating a machine learning model based on techniques such as linear regression, logistic regression, support vector machines (SVM), convolutional neural networks (CNN), or deep learning, wherein the distribution of cfDNA distances or a set of variables derived from it, such as the locations of some of the peaks of the said distribution, is represented as a vector characterising each cfDNA sample;
(c) training the said machine learning model using the frequency distributions of cfDNA distances or a set of variables derived from the said distribution of cfDNA distances for one or more healthy and diseased conditions; and
(d) performing the classification of a given subject using the distribution of cfDNA distances or a set of variables derived from the said distribution using the said machine learning model.

43. The method of claim 1 for the use in determining a subject's disease state using Fourier transform (FT), discrete Fourier transform (DFT), fast Fourier transform (FFT) or other Fourier transform-based algorithms for the analysis of nucleosome spacing genome-wide or in genomic regions of interest, the method comprising:
(a) determining one or more pronounced frequencies of the Fourier transform-based transformation of the distribution of nucleosome spacing for a subject in at least one timepoint;
(b) computing the corresponding NRL values as the values inverse to the said frequencies, and
(c) comparing the determined values of said NRLs to at least one set of reference NRL values;
wherein a time-dependent change of the Fourier transform-based transformation amplitudes associated with said frequencies and NRL values indicates a presence or absence of a disease or a specific state of healthy functioning.

44. The method of claim 43, wherein NRL values with the largest peaks of Fourier-transform amplitude for cfDNA from healthy people are about 200 bp and about 182 bp, and Fourier transform-based NRL value for cfDNA from breast cancer patients is about 182 bp (lacking the NRL value around 200 bp in the case of cancer).

45. The method of claim 31, wherein the sets of reference NRL values and frequency distributions of cfDNA distances are from:
(i) a healthy cohort;
(ii) a diseased cohort;
(iii) cohorts of people with different ages;
(iv) cohorts of people with different ethnicities;
(v) cohorts of people with different weight or body mass index (BMI);
(vi) cohorts of people with different lifestyle; and/or
(vii) cohorts of people with different diet.

46. The method of claim 31, wherein the disease is cancer and/or the specific state of healthy functioning is characterised by person's age, BMI, lifestyle or diet.
